(19) United States
(12) Patent Application Publication
(10) Pub. No.: US 2026/0355225 A1
(43) Pub. Date: Nov. 12, 2026

(54) USE OF D-XYLOSE TO STIMULATE GLYCOSAMINOGLYCAN BIOSYNTHESIS

(71) Applicant: Antony CHEUDJEU, Massy (FR)

(72) Inventor: Antony CHEUDJEU, Massy (FR)

(21) Appl. No.: 19/134,678

(22) PCT Filed: Dec. 4, 2023
(86) PCT No.: PCT/EP2023/084075

§ 371 Date: May 30, 2025

(30) Foreign Application Priority Data
Dec. 5, 2022 (FR) FR2212766

(51) Int. Cl. A61K 31/7004; A61K 36/04; A61K 36/185; A61K 36/19; A61K 36/282; A61K 36/355; A61K 36/39; A61K 36/899

(52) U.S. Cl. CPC A61K 31/7004; A61K 36/04; A61K 36/185; A61K 36/19; A61K 36/282; A61K 36/355; A61K 36/39; A61K 36/899

(57) ABSTRACT

The invention relates to a formulation comprising at least one compound selected from D-xylose, esters thereof, oligosaccharides comprising D-xylose, and/or β-D-xylosides, for use in stimulating the biosynthesis of glycosaminoglycans, preferably at least one selected from heparan sulfate, dermatan sulfate and chondroitin sulfate, in a human patient, by administering between 1 and 10 doses per day of a composition having a mass of 500 mg to 10 g of said compound. Preferably, said formulation is for use in the prevention or treatment of infections with viruses using syndecans and/or glypicans as cell receptors, such as SARS-CoV-2 or HIV, or in the prevention or treatment of type 2 diabetes or of insulin resistance. The invention also relates to the use of an oral or nasal food supplement composition in healthy subjects to stimulate glycosaminoglycan biosynthesis.

The invention relates to a formulation comprising at least one compound selected from D-xylose, esters thereof, oligosaccharides comprising D-xylose, and/or β-D-xylosides, for use in stimulating the biosynthesis of glycosaminoglycans, preferably at least one selected from heparan sulfate, dermatan sulfate and chondroitin sulfate, in a human patient, by administering between 1 and 10 doses per day of a composition having a mass of 500 mg to 10 g of said compound. Preferably, said formulation is for use in the prevention or treatment of infections with viruses using syndecans and/or glypicans as cell receptors, such as SARS-CoV-2 or HIV, or in the prevention or treatment of type 2 diabetes or of insulin resistance. The invention also relates to the use of an oral or nasal food supplement composition in healthy subjects to stimulate glycosaminoglycan biosynthesis.

### BACKGROUND ART

The SARS-CoV-2 pandemic currently affecting the whole world has proved particularly dangerous in the elderly and in smokers, with high comorbidity in patients already suffering from chronic illnesses such as type 2 diabetes, cancer, chronic respiratory disease, obesity, and hypertension.

At the same time, several studies have shown that inflammation of the lungs in general and type 2 diabetes in particular are accompanied by a degradation of the glycosaminoglycans (GAGs), notably of the heparan sulfate (HS). Several studies have also shown the importance of countering HS degradation in lung infections and type 2 diabetes.

D-xylose is only commercially available for the D-xylose test, in which this molecule is ingested orally to measure intestinal absorption in patients. No other medicinal uses have been reported to date.

Only two patent families disclose its use in the cosmetics/pharmaceutical field: the WO1999024009A1 family of 1999 for its use in cosmetic products and the WO1999048361A1 family of 1998 for the treatment of diseases of the upper respiratory tract. However, these two patent families make no mention of the antiviral properties described by the Applicant.

Several in vitro studies of SARS-CoV-2 have demonstrated the involvement of GAGs, more specifically HS, in viral attachment. Additionally, the involvement of HS in the viral attachment of more than a dozen envelope viruses has been reported in the literature (see Cheudjeu, 2021).

With regard to this involvement, the conclusions reached by leading laboratories in the field following in vitro studies on the interaction between SARS-CoV-2 and GAGs, were that “HS promotes infection” (Claussen, T. M, 2020), arriving at this consensus. One notable example that can be cited is a study by a team of Chinese researchers, who confirmed that there was indeed a correlation between the severity of COVID-19 and D-xylose (Zhong et al, 2021). However, they concluded that the suggestion to use D-xylose as a drug against COVID-19 was not supported by their data.

In this way, the formulation developed by the Applicant runs counter to the consensus of the scientific community as a whole, and nevertheless shows convincing results, as shown in the examples.

Additionally, however, taking D-xylose in absorption tests is generally associated with side effects such as laxative effects in adults, diarrhea, nausea, and vomiting.

For these reasons, it does not appear conceivable for a skilled person to develop such a composition in the indications set out in the invention.

Additionally, the state of the art does not disclose a dosage regimen for limiting these side effects.

Surprisingly, the Applicant has thus been able to develop a formulation based on D-xylose or one of the derivatives thereof, which makes it possible to act on viruses using syndecans and/or glypicans as cell receptors, while solving the additional technical problem of limiting the side effects associated with D-xylose or the derivatives thereof.

More specifically, the invention relates to novel uses of D-xylose or of products derived from or containing D-xylose to stimulate the biosynthesis of glycosaminoglycans with D-xylose as their chain-initiating molecule (heparan sulfates, dermatan sulfates, chondroitin sulfates), while reducing the side effects associated with the use thereof. The novel uses relate to the prevention or treatment of viral infections using syndecans and/or glypicans as cell receptors.

The invention thus describes uses of D-xylose, of the derivatives thereof or of products containing D-xylose, made possible by applying an effective dosage to limit the side effects of the use thereof in a patient or consumer.

The common inventive concept in the context of this invention is the use of at least one compound selected from D-xylose, esters thereof, oligosaccharides comprising D-xylose, and/or β-D-xylosides, to increase glycosaminoglycan synthesis, in a healthy human, or a patient at risk of or suffering from infections with viruses using syndecans and/or glypicans as cell receptors, preferably said virus being selected from HSV-1, HSV-2, HPV-16, HPV-31, HVB, HVC, HIV-1, HTLV-1, SARS-CoV-2, HCMV, DENV-1, and DENV-2, in the treatment of COVID-19, AIDS, type 2 diabetes, or insulin resistance. These compounds are used in such a way as to limit the side effects of the use thereof for a patient or consumer.

### DESCRIPTION OF THE INVENTION

According to a first aspect, the invention relates to a formulation comprising at least one compound selected from D-xylose, esters thereof, oligosaccharides comprising D-xylose, and/or β-D-xylosides, in a physiologically acceptable medium for use in a human patient in stimulating the biosynthesis of glycosaminoglycans.

The mechanism of action of this formulation is to prevent, through the administration of D-xylose or one of the derivatives thereof, the replication of the virus, and thus reduce the symptoms and biological markers associated with the severity of the viral infection, notably cough, inflammation, etc. Making the infection benign (asymptomatic). After administering D-xylose or one of the derivatives thereof, the latter competes with the viral load by binding to the front central protein of the viruses, to initiate the production of glycosaminoglycans (heparan sulfate, dermatan sulfate and chondroitin sulfate).

Preferably, the invention relates to a formulation comprising between 500 mg and 10 g of at least one compound selected from D-xylose, esters thereof, oligosaccharides comprising D-xylose, and/or β-D-xylosides, in a physiologically acceptable medium, for use in a human patient in stimulating the biosynthesis of glycosaminoglycans.

Preferably, the invention relates to a formulation comprising at least one compound selected from D-xylose, esters thereof, oligosaccharides comprising D-xylose, and/or β-D-xylosides, for use in a human patient in stimulating the biosynthesis of glycosaminoglycans by administering between 1 and 10 doses per day of a composition having a mass of 500 mg to 10 g of said at least one compound in a physiologically acceptable medium.

Taking small doses makes it possible to reduce the side effects, notably the laxative effects, nausea, and vomiting.

Preferably, the biosynthesis of glycosaminoglycans is increased by at least 1%, preferably by at least 5%, preferably by at least 10%, preferably by at least 20%, preferably by at least 30%, preferably by at least 40%, preferably by at least 50%, preferably by at least 60%, preferably by at least 70%, preferably by at least 80%, preferably by at least 90%, preferably by at least 100%, preferably by at least 150%, preferably by at least 200%, preferably by at least 500%, preferably by at least 1000%.

Preferably, said glycosaminoglycan is a sulfated glycosaminoglycan.

Preferably, said glycosaminoglycans are at least one selected from heparan sulfate, dermatan sulfate, and chondroitin sulfate.

According to one embodiment, said formulation is for use in the prevention or treatment of infection with viruses using syndecans and/or glypicans as cell receptors, preferably said virus being selected from HSV-1, HSV-2, HPV-16, HPV-31, HVB, HVC, HIV-1, HTLV-1, SARS-CoV-2, HCMV, DENV-1, and DENV-2.

Even more preferably, said formulation is for use in the prevention or treatment of SARS-CoV-2 infection.

Even more preferably, said formulation is for use in the prevention or treatment of HIV-1 infection.

According to one embodiment, said formulation is for use in the prevention or treatment of COVID-19.

According to one embodiment, said formulation is for use in the prevention or treatment of type 2 diabetes.

According to one embodiment, said formulation is for use in the prevention or treatment of insulin resistance.

In one embodiment, the number of administrations per day is 1.

Preferably, the number of administrations per day is between 2 and 10.

Even more preferably, the number of administrations per day is 2, or 3, or 4, or 5, or 6, or 7, or 8, or 9, or 10.

The number of administrations per day can be increased according to the risk of developing the disease, or according to its severity.

Preferably, said formulation for use according to the invention is in a form suitable for oral, nasal or parenteral administration.

Preferably, the composition is in a form suitable for parenteral administration via the subcutaneous, intradermal, intravenous, or intramuscular route.

Preferably, the composition for oral administration is formulated as capsules, gelcaps, tablets, effervescent tablets, powders, granules, oral solutions, or suspensions.

Preferably, the composition for nasal administration is formulated as an aerosol solution.

Preferably, the composition is in a form suitable for oral administration by administering between 1 and 10 doses per day of a composition having a mass of 500 mg to 10 g of at least one compound selected from D-xylose, esters thereof, oligosaccharides comprising D-xylose, and/or β-D-xylosides.

Preferably, the composition is in a form suitable for oral administration by administering between 1 and 10 doses per day of a composition having a mass of 500 mg to 10 g of D-xylose.

Preferably, the composition is in a form suitable for parenteral administration by administering between 1 and 10 doses per day of a composition having a mass of 1 g to 10 g of at least one compound selected from D-xylose, esters thereof, oligosaccharides comprising D-xylose, and/or β-D-xylosides.

Preferably, the composition is in a form suitable for parenteral administration by administering between 1 and 10 doses per day of a composition having a mass of 1 g to 10 g of D-xylose.

Preferably, the composition is in a form suitable for nasal administration by administering between 1 and 10 doses per day of a composition having a mass of 500 mg to 10 g of at least one compound selected from D-xylose, esters thereof, oligosaccharides comprising D-xylose, and/or β-D-xylosides.

Preferably, the composition is in a form suitable for nasal administration by administering between 1 and 10 doses per day of a composition having a mass of 500 mg to 10 g of D-xylose.

Preferably, said at least one compound selected from D-xylose, esters thereof, oligosaccharides comprising D-xylose, and/or β-D-xylosides, is D-xylose.

According to one embodiment, D-xylose is present in the composition in the form of plant extracts comprising D-xylose.

Alternatively, said plant extracts are selected from extracts of honeysuckle, Japanese honeysuckle, green chiretta, red algae, water bindweed, birch leaf, bark or sap, a species of the genus Artemisia, rice stem.

In another alternative, D-xylose is derived from lignocellulose from plant material. Preferably, D-xylose is supplied in the form of maize stalk hemicellulose hydrolysates.

According to one embodiment, the composition further comprises at least one other acceptable active pharmaceutical ingredient and/or at least one excipient and/or at least one acceptable pharmaceutical carrier and/or any pharmaceutically acceptable compound.

Preferably, said at least one other pharmaceutical ingredient is an antiviral active ingredient.

Preferably, said at least one other pharmaceutical ingredient is an active ingredient for the prevention or treatment of COVID-19.

Preferably, said at least one other pharmaceutical ingredient is an active ingredient for the prevention or treatment of type 2 diabetes.

Preferably, said at least one other pharmaceutical ingredient is an active ingredient for the prevention or treatment of insulin resistance.

Preferably, the composition further comprises an active agent for accelerating the transfer of said at least one compound selected from D-xylose, esters thereof, oligosaccharides comprising D-xylose, and/or β-D-xylosides, preferably D-xylose, to the cells, said active agent preferably being insulin.

According to one embodiment of the invention, the formulation according to the invention also comprises at least one antibiotic.

According to one embodiment of the invention, the formulation according to the invention comprises a hormone accelerating the transport of D-xylose or the derivatives thereof into the cell, like insulin, or also any pharmaceutically acceptable enzymes facilitating the transport of D-xylose into the cell, for example xylose transporters.

According to one embodiment of the invention, the composition according to the invention also comprises a compound for reducing at least one side effect of D-xylose intake, preferably a compound for reducing vomiting or diarrhea.

According to one embodiment of the invention, the composition according to the invention also comprises vitamin C.

According to a second aspect, the invention relates to the use of an oral or nasal food supplement composition in healthy subjects comprising at least one compound selected from D-xylose, esters thereof, oligosaccharides comprising D-xylose, and/or β-D-xylosides in a physiologically acceptable medium to stimulate the biosynthesis of glycosaminoglycans.

Preferably, said glycosaminoglycan is a sulfated glycosaminoglycan.

Preferably, said glycosaminoglycans are at least one selected from heparan sulfate, dermatan sulfate, and chondroitin sulfate.

Preferably, the invention relates to the use of an oral or nasal food supplement composition for healthy subjects comprising between 500 mg and 10 g of at least one compound selected from D-xylose, esters thereof, oligosaccharides comprising D-xylose, and/or β-D-xylosides, in a physiologically acceptable medium, in stimulating the biosynthesis of glycosaminoglycans.

Preferably, said glycosaminoglycan is a sulfated glycosaminoglycan.

Preferably, said glycosaminoglycans are at least one selected from heparan sulfate, dermatan sulfate, and chondroitin sulfate.

Preferably, said at least one compound selected from D-xylose, esters thereof, oligosaccharides comprising D-xylose, and/or β-D-xylosides, is D-xylose.

Preferably, the food supplement composition is in a form suitable for oral administration by administering between 1 and 10 doses per day of a composition having a mass of 500 mg to 10 g of at least one compound selected from D-xylose, esters thereof, oligosaccharides comprising D-xylose, and/or β-D-xylosides

Preferably, the food supplement composition is in a form suitable for oral administration by administering between 1 and 10 doses per day of a composition having a mass of 500 mg to 10 g of D-xylose.

Preferably, the food supplement composition is in a form suitable for nasal administration by administering between 1 and 10 doses per day of a composition having a mass of 500 mg to 10 g of at least one compound selected from D-xylose, esters thereof, oligosaccharides comprising D-xylose, and/or β-D-xylosides.

Preferably, the food supplement composition is in a form suitable for nasal administration by administering between 1 and 10 doses per day of a composition having a mass of 500 mg to 10 g of D-xylose.

Preferably, the biosynthesis of glycosaminoglycans is increased by at least 1%, preferably by at least 5%, preferably by at least 10%, preferably by at least 20%, preferably by at least 30%, preferably by at least 40%, preferably by at least 50%, preferably by at least 60%, preferably by at least 70%, preferably by at least 80%, preferably by at least 90%, preferably by at least 100%, preferably by at least 150%, preferably by at least 200%, preferably by at least 500%, preferably by at least 1000%.

In one embodiment, the food supplement is formulated for oral administration as capsules, gelcaps, tablets, effervescent tablets, powders, granules, oral solutions, or suspensions.

Preferably, the food supplement for nasal administration is formulated as an aerosol solution.

According to one embodiment, the food supplement is formulated for oral intake in a food product, beverage, food additive or dairy product, containing said food supplement.

In one embodiment, the number of administrations per day is 1.

Preferably, the number of administrations per day is between 2 and 10.

Even more preferably, the number of administrations per day is 2, or 3, or 4, or 5, or 6, or 7, or 8, or 9, or 10.

The number of administrations per day can be increased according to the needs of the consumer.

Preferably, D-xylose is present in the food supplement composition in the form of plant extracts comprising D-xylose.

Alternatively, said plant extracts are selected from extracts of honeysuckle, Japanese honeysuckle, green chiretta, red algae, water bindweed, birch leaf, bark or sap, a species of the genus Artemisia, rice stem.

In another alternative, D-xylose is derived from lignocellulose from plant material. Preferably, D-xylose is supplied in the form of maize stalk hemicellulose hydrolysates.

According to one embodiment of the invention, the food supplement composition according to the invention also comprises vitamin C.

According to one embodiment of the invention, the food supplement composition according to the invention also comprises galactose.

According to a third aspect, the invention relates to a therapeutic method for stimulating the biosynthesis of glycosaminoglycans comprising administering to a human patient between 1 and 10 doses per day of a composition having a mass of 500 mg to 10 g of at least one compound selected from D-xylose, esters thereof, oligosaccharides comprising D-xylose, and/or β-D-xylosides in a physiologically acceptable medium.

Preferably, said glycosaminoglycan is a sulfated glycosaminoglycan.

Preferably in this therapeutic method, said glycosaminoglycan is at least one selected from heparan sulfate, dermatan sulfate and chondroitin sulfate.

Preferably, said at least one compound selected from D-xylose, esters thereof, oligosaccharides comprising D-xylose, and/or β-D-xylosides, is D-xylose.

Preferably, the biosynthesis of glycosaminoglycans is increased by at least 1%, preferably by at least 5%, preferably by at least 10%, preferably by at least 20%, preferably by at least 30%, preferably by at least 40%, preferably by at least 50%, preferably by at least 60%, preferably by at least 70%, preferably by at least 80%, preferably by at least 90%, preferably by at least 100%, preferably by at least 150%, preferably by at least 200%, preferably by at least 500%, preferably by at least 1000%.

According to one embodiment of this therapeutic method, said formulation is for use in the prevention or treatment of infection with viruses using syndecans and/or glypicans as cell receptors, preferably said virus being selected from HSV-1, HSV-2, HPV-16, HPV-31, HVB, HVC, HIV-1, HTLV-1, SARS-CoV-2, HCMV, DENV-1, and DENV-2.

Even more preferably in this therapeutic method, said formulation is for use in the prevention or treatment of SARS-CoV-2 infection.

Even more preferably in this therapeutic method, said formulation is for use in the prevention or treatment of HIV-1 infection.

According to one embodiment, this therapeutic method is in the prevention or treatment of COVID-19.

According to one embodiment, this therapeutic method is in the prevention or treatment of type 2 diabetes.

According to one embodiment, this therapeutic method is in the prevention or treatment of insulin resistance.

Preferably, the number of administrations per day is between 2 and 10.

Even more preferably, the number of administrations per day is 2, or 3, or 4, or 5, or 6, or 7, or 8, or 9, or 10.

The number of administrations per day can be increased according to the risk of developing the disease, or according to its severity.

Preferably, D-xylose is present in the composition in the form of plant extracts comprising D-xylose.

Preferably in this therapeutic method, said composition according to the invention is in a form suitable for oral, nasal or parenteral administration.

Preferably in this therapeutic method, the composition is in a form suitable for subcutaneous, intradermal, intravenous or intramuscular parenteral administration.

Preferably in this therapeutic method, the composition is in a form suitable for oral administration by administering between 1 and 10 doses per day of a composition having a mass of 500 mg to 10 g of at least one compound selected from D-xylose, esters thereof, oligosaccharides comprising D-xylose, and/or β-D-xylosides.

Preferably in this therapeutic method, the composition is in a form suitable for oral administration by administering between 1 and 10 doses per day of a composition having a mass of 500 mg to 10 g of D-xylose.

Preferably in this therapeutic method, the composition is in a form suitable for parenteral administration by administering between 1 and 10 doses per day of a composition having a mass of 1 g to 10 g of at least one compound selected from D-xylose, esters thereof, oligosaccharides comprising D-xylose, and/or β-D-xylosides.

Preferably in this therapeutic method, the composition is in a form suitable for parenteral administration by administering between 1 and 10 doses per day of a composition having a mass of 1 g to 10 g of D-xylose.

Preferably in this therapeutic method, the composition is in a form suitable for nasal administration by administering between 1 and 10 doses per day of a composition having a mass of 500 mg to 10 g of at least one compound selected from D-xylose, esters thereof, oligosaccharides comprising D-xylose, and/or β-D-xylosides.

Preferably, the composition is in a form suitable for nasal administration by administering between 1 and 10 doses per day of a composition having a mass of 500 mg to 10 g of D-xylose.

Alternatively, said plant extracts are selected from extracts of honeysuckle, Japanese honeysuckle, green chiretta, red algae, water bindweed, birch leaf, bark or sap, a species of the genus Artemisia, rice stem.

In another alternative, D-xylose is derived from lignocellulose from plant material. Preferably, D-xylose is supplied in the form of maize stalk hemicellulose hydrolysates.

Preferably, the composition further comprises an active agent for accelerating the transfer of said at least one compound selected from D-xylose, esters thereof, oligosaccharides comprising D-xylose, and/or β-D-xylosides, preferably D-xylose, to the cells, said active agent preferably being insulin.

According to one embodiment of the invention, the composition according to the invention also comprises at least one antibiotic.

According to one embodiment of the invention, the formulation according to the invention comprises a hormone accelerating the transport of D-xylose or the derivatives thereof into the cell, like insulin, or also any pharmaceutically acceptable enzymes facilitating the transport of D-xylose into the cell, for example xylose transporters.

According to one embodiment of the invention, the composition according to the invention also comprises a compound for reducing at least one side effect of D-xylose intake, preferably a compound for reducing vomiting or diarrhea.

According to one embodiment of the invention, the composition according to the invention also comprises vitamin C.

According to a fourth aspect, the invention relates to the use of at least one compound selected from D-xylose, esters thereof, oligosaccharides comprising D-xylose, and/or β-D-xylosides, in a physiologically acceptable medium for the preparation of a drug used for stimulating the biosynthesis of glycosaminoglycans in a human patient.

Preferably, the invention relates to the use of at least one compound selected from D-xylose, esters thereof, oligosaccharides comprising D-xylose, and/or β-D-xylosides, for the preparation of a drug used for stimulating the biosynthesis of glycosaminoglycans in a human patient, and wherein the administration amount of said at least one compound is between 500 mg and 10 g in a physiologically acceptable medium per administration, the number of administrations per day being between 1 and 10 administrations.

Preferably, said glycosaminoglycan is a sulfated glycosaminoglycan.

Preferably, said glycosaminoglycans are at least one selected from heparan sulfate, dermatan sulfate, and chondroitin sulfate.

Preferably, said at least one compound selected from D-xylose, esters thereof, oligosaccharides comprising D-xylose, and/or β-D-xylosides, is D-xylose.

According to one embodiment, said use relates to the preparation of a drug in the prevention or treatment of infection with viruses using syndecans and/or glypicans as cell receptors, preferably said virus being selected from HSV-1, HSV-2, HPV-16, HPV-31, HVB, HVC, HIV-1, HTLV-1, SARS-CoV-2, HCMV, DENV-1, and DENV-2.

According to one embodiment, said use relates to the preparation of a drug in the prevention or treatment of SARS-CoV-2 infection.

According to one embodiment, said use relates to the preparation of a drug in the prevention or treatment of HIV-1 infection.

According to one embodiment, said use relates to the preparation of a drug in the prevention or treatment of COVID-19.

According to one embodiment, said use relates to the preparation of a drug in the prevention or treatment of type 2 diabetes.

According to one embodiment, said use relates to the preparation of a drug in the prevention or treatment of insulin resistance.

Preferably, the biosynthesis of glycosaminoglycans is increased by at least 1%, preferably by at least 5%, preferably by at least 10%, preferably by at least 20%, preferably by at least 30%, preferably by at least 40%, preferably by at least 50%, preferably by at least 60%, preferably by at least 70%, preferably by at least 80%, preferably by at least 90%, preferably by at least 100%, preferably by at least 150%, preferably by at least 200%, preferably by at least 500%, preferably by at least 1000%.

Preferably, the number of administrations per day is between 2 and 10.

Even more preferably, the number of administrations per day is 2, or 3, or 4, or 5, or 6, or 7, or 8, or 9, or 10.

The number of administrations per day can be increased according to the risk of developing the disease, or according to its severity.

According to one embodiment, said use relates to the preparation of a drug in a form suitable for oral, nasal or parenteral administration.

Preferably, said use relates to the preparation of a drug in a form suitable for parenteral administration by the subcutaneous, intradermal, intravenous or intramuscular route.

Preferably, said use relates to the preparation of a drug for oral administration in the form of capsules, gelcaps, tablets, effervescent tablets, powders, granules, oral solutions, or suspensions.

Preferably, the composition for nasal administration is formulated as an aerosol solution.

Preferably, said use relates to the preparation of a drug for oral administration, the administration of which is performed by administering between 1 and 10 doses per day of a composition having a mass of 500 mg to 10 g of at least one compound selected from D-xylose, esters thereof, oligosaccharides comprising D-xylose, and/or β-D-xylosides.

Preferably, said use relates to the preparation of a drug for oral administration, the administration of which is carried out by administering between 1 and 10 doses per day of a composition having a mass of 500 mg to 10 g of D-xylose.

Preferably, said use relates to the preparation of a drug for parenteral administration, the administration of which is performed by administering between 1 and 10 doses per day of a composition having a mass of 1 g to 10 g of at least one compound selected from D-xylose, esters thereof, oligosaccharides comprising D-xylose, and/or β-D-xylosides,

Preferably, said use relates to the preparation of a drug for parenteral administration, the administration of which is carried out by administering between 1 and 10 doses per day of a composition having a mass of 1 g to 10 g of D-xylose.

Preferably, said use relates to the preparation of a drug for nasal administration, the administration of which is performed by administering between 1 and 10 doses per day of a composition having a mass of 500 mg to 10 g of at least one compound selected from D-xylose, esters thereof, oligosaccharides comprising D-xylose, and/or β-D-xylosides.

Preferably, said use relates to the preparation of a drug for nasal administration, the administration of which is performed by administering between 1 and 10 doses per day of a composition having a mass of 500 mg to 10 g of D-xylose.

According to one embodiment, said drug prepared in the form of a composition further comprises at least one other acceptable active pharmaceutical ingredient and/or at least one excipient and/or at least one acceptable pharmaceutical carrier and/or any pharmaceutically acceptable compound.

Preferably, said at least one other pharmaceutical ingredient is an antiviral active ingredient.

Preferably, said at least one other pharmaceutical ingredient is an active ingredient for the prevention or treatment of COVID-19.

Preferably, said at least one other pharmaceutical ingredient is an active ingredient for the prevention or treatment of type 2 diabetes.

Preferably, said at least one other pharmaceutical ingredient is an active ingredient for the prevention or treatment of insulin resistance.

Preferably, D-xylose is present in the composition in the form of plant extracts comprising D-xylose.

Alternatively, said plant extracts are selected from extracts of honeysuckle, Japanese honeysuckle, green chiretta, red algae, water bindweed, birch leaf, bark or sap, a species of the genus Artemisia, rice stem.

In another alternative, D-xylose is derived from lignocellulose from plant material. Preferably, D-xylose is supplied in the form of maize stalk hemicellulose hydrolysates.

According to one embodiment of the invention, the composition according to the invention also comprises the addition of antibiotics.

According to one embodiment of the invention, the composition comprises a hormone accelerating the transport of D-xylose or the derivatives thereof into the cell, like insulin, or also any pharmaceutically acceptable enzymes facilitating the transport of D-xylose into the cell, for example xylose transporters.

According to one embodiment of the invention, the composition according to the invention also comprises a compound for reducing at least one side effect of D-xylose intake, preferably a compound for reducing vomiting or diarrhea.

According to one embodiment of the invention, the composition according to the invention also comprises the addition of vitamin C.

Preferably, the composition further comprises an active agent for accelerating the transfer of said at least one compound selected from D-xylose, esters thereof, oligosaccharides comprising D-xylose, and/or β-D-xylosides, preferably D-xylose, to the cells, said active agent preferably being insulin.

### FIGURES

FIG. 1 shows the results of IC50 concentrations for the two modes of administration: (1) Co-administration of D-xylose and SARS-CoV-2, IC50=95.52 mM (2) Pre-incubation of D-xylose 6 h prior to SARS-CoV-2 infection IC50=26.22 mM.

FIG. 2 shows the results for remdesivir used as a positive control in antiviral assays.

FIG. 3 shows the results of the percentage inhibition of HIV-1 NL4-3 according to D-xylose concentration alone, with calculation of the median inhibitory concentration (IC50) and median cytotoxic concentration (TC50).

FIG. 4 shows the results of the percentage inhibition of HIV-1 NL4-3 according to the concentration of D-xylose+insulin, with calculation of the median inhibitory concentration (IC50) and median cytotoxic concentration (TC50).

FIG. 5 shows the percentage inhibition of HIV-1 NL4-3 according to the AZT concentration (used as a control), with calculation of the median inhibitory concentration (IC50).

FIG. 6 shows the results of the immunofluorescence assay for 96-well plates in the same layout as shown in [Table 11]

### DEFINITIONS

### D-Xylose and Mechanism of Action

D-xylose is a pentose, present in the bioactive form in the human body, which is also found in nature, in wood bark and in certain plants. This molecule has been approved by the FDA for use in the D-xylose test.

In the body, D-xylose is used in sulfated glycosaminoglycans (GAGs), which are long linear chains of disaccharide repeating units present in the extracellular matrix (ECM), on the surface of endothelial cells, epithelial cells, fibroblasts, macrophages and hepatocytes (R. R. Vivès, Héparanes sulfate: Structure, fonctions, régulation, 11 Apr. 2020). There are five types of sulfated GAGs: heparan sulfates (HS), heparins (Hep), chondroitin sulfates (CS), dermatan sulfates (DS) and keratan sulfates (KS). The sulfated GAGs are bound to a core protein (syndecans, glypicans, decorins, etc.) by a covalent bond, forming proteoglycans (PGs).

The main core proteins of PGs in the extracellular matrix are perlecan (with HS-type chains), agrin (heparan sulfate proteoglycan [HSPG]), aggrecan (chondroitin sulfate proteoglycan [CSPG]/dermatan sulfate proteoglycan [DSPG]) and decorin.

The central membrane proteins of PGs are classified into two categories: syndecans and glypicans. The syndecans are transmembrane proteins, unlike the glypicans, which are entirely extracellular proteins (fixed to the cell membrane by a glycosyl-phosphatidyl inositol (GPI)-type anchor. The membrane PGs include HSPGs, CSPGs and DSPGs, but are mainly HSPGs. The HSPGs are found on the surface of many cell types, including endothelial cells, epithelial cells, fibroblasts and neural tissue (R. R. Vivès, Héparanes sulfate: Structure, fonctions, régulation, 11 Apr. 2020 & S. Sarrazin, W. C. Lamanna, J. D. Esko, Heparan sulfate proteoglycans, Cold Spring Harb. Perspect. Biol. 3 (2011))

With the exception of KS, all the other sulfated GAGs are bound to the core proteins by an identical binding region, consisting of the trisaccharide sequence xylose-galactose-galactose. Biosynthesis of the HS chain (and also of the CS, DS and Hep chains) begins with the fixing of a D-xylose molecule to specific serine and occasionally threonine residues on the core protein (syndecan, glypican, decorin, etc.). This initiation of HS/CS/DS biosynthesis is carried out either by the enzymes xylosyltransferase 1 and xylosyltransferase 2 (R. R. Vivès, Héparanes sulfate: Structure, fonctions, régulation, 11 Apr. 2020 & S. Sarrazin, W. C. Lamanna, J. D. Esko, Heparan sulfate proteoglycans, Cold Spring Harb. Perspect. Biol. 3 (2011)) or directly by free molecules (unlike UDP-Xyl) of D-xylose (Cheudjeu, 2022).

After the D-xylose molecule is in place, the bonding of two galactose molecules follows. The chain thus formed constitutes the binding region. This is then completed by a repeat of the same basic unit specific to each type of sulfated GAG (HS/CS/DS/Hep).

The basic unit of HS is a D-glucuronic acid linked to N-acetylglucosamine. The basic unit of CS is an N-acetylgalactosamine linked to a D-glucuronic acid. Hyaluronic acid is the only non-sulfated GAG; this type of glycosaminoglycan is not bound to a central protein. The disaccharides of which it is composed are themselves made up of D-glucuronic acid and D-N-acetylglucosamine (R. R. Vivès, Héparanes sulfate: Structure, fonctions, régulation, 11 Apr. 2020).

The HSPGs are rapidly recycled and renewed on the surface of the cell; their half-life is about 2-3 h (M. Egeberg, R. Kjeken, S. O. Kolset, T. Berg, K. Prydz, Internalization and stepwise degradation of heparan sulfate proteoglycans in rat hepatocytes, Biochim. Biophys. Acta 1541 (2001) 135-149). Degradation of HSPGs on the surface of the cells depends on the action of several extracellular and lysosomal enzymes, in particular heparanase, which cleaves the HS chains (R. R. Vivès, Héparanes sulfate: Structure, fonctions, régulation, 11 Apr. 2020 & S. Sarrazin, W. C. Lamanna, J. D. Esko, Heparan sulfate proteoglycans, Cold Spring Harb. Perspect. Biol. 3 (2011)).

D-xylose is therefore the element linking these GAGs to the core proteins, and has only one position. This role of D-xylose in HS/CS/DS/Hep biosynthesis indicates that the amount of D-xylose in the body directly influences the amount of these GAGs present in that body.

According to the invention, “D-xylose derivatives” means D-xylose esters, oligosaccharides comprising D-xylose, and/or β-D-xylosides.

### Type 2 Diabetes, Insulin Resistance and Mechanism of Action

Type 2 diabetes mellitus (T2DM) is a metabolic disease that is often one of the symptoms of certain viral infections, as is the case with SARS-CoV-2, HIV-1, HVC, etc. (Cheudjeu 2021).

Several explanations for the onset of T2DM during viral infections have been given, such as: inflammation (cytokine storm) due to infections that cause damage to pancreatic β-cells, leading to insulin homeostasis. However, if this were the only explanation, insulin supplementation would definitely be sufficient, but this is not always the case. This inability of insulin to reduce blood glucose levels is referred to as insulin resistance and is widely reported in viral infections that use core proteins as receptors on the surface of the cells: SARS-CoV-2 (Govender N et al., 2021: PMID: 33849817), HVC (Desai D. V et al., 2010: https://doi.org/10.1096/fasebj.24.1_supplement.659.4), HIV-1 (Pedro M. N et al., 2018: PMID: 30233499), etc. Insulin resistance is also often observed in some obese patients with T2DM (Kahn, B. B., and Flier, J. S, 2000). The present invention describes for the first time how insulin resistance is linked to the ability of D-xylose to stimulate GAGs. Most of the sugars making up the GAG chains are glucose metabolites (Cheudjeu 2022).

In the event of insufficient D-xylose in the body (or in the case of certain viral infections), one of the first consequences is an increase in blood sugar levels. Indeed, some serine sites that should be occupied by D-xylose on the core proteins (syndecans, glypicans, etc.) to initiate GAG production (HS/Hep/CS/DS) are free due to a lack of D-xylose (or are occupied due to virus glycosylation in the case of certain viral infections), thus preventing the initiation of GAG biosynthesis that should take place at these sites. The sugars that should be used in the production of these GAGs (D-glucuronic acid, galactose, N-acetylglucosamine, N-acetylgalactosamine) are found in the bloodstream (Cheudjeu 2020; Cheudjeu 2021; Cheudjeu 2022).

A 2011 study showed that the sulfated GAGs were altered during type 2 diabetes, with HS and chondroitin/dermatan sulfate (CS/DS) levels decreased by about 14% (D. Joladarashi, P. V. Salimath, N. D. Chilkunda, Diabetes results in structural alteration of chondroitin sulfate/dermatan sulfate in the rat kidney: effects on the binding to extracellular matrix components, Glycobiology 21 (2011) 960-972).

Several other studies have reported HS degradation during diabetes (L. M. Hiebert, J. Han, A. K. Mandal, Glycosaminoglycans, hyperglycemia, and disease, Antioxid. Redox Signal. 21 (2014) 1032-1043 & L. M. Hiebert, Proteoglycans and diabetes, Curr. Pharm. Des. 23 (2017) 1500-1509).

Also, given the half-life of the GAGs on the surface of the cell, which is 2-3 hours, this degradation contributes to the accumulation of HS/CS/DS chains, which are the source of certain cardiovascular diseases. Other studies have already shown that N-acetylglucosamine levels increase during type 2 diabetes and that N-acetylglucosamine can be used as a biomarker for type 2 diabetes (Z. Wang, K. Park, F. Comer, L. C. Hsieh-Wilson, C. D. Saudek, G. W. Hart, Site-specific GlcNAcylation of human erythrocyte proteins: potential biomarker(s) for diabetes, Diabetes 58 (2009) 309-317, & L. Wells, K. Vosseller, G. W. Hart, A role for N-acetylglucosamine as a nutrient sensor and mediator of insulin resistance, Cell. Mol. Life Sci. 60 (2003) 222-228.

Another consequence is an increase in other types of GAGs, such as hyaluronic acid (HA). Indeed, hyaluronic acid not bound to a central protein can act to fix excess sugar not used for HS/CS/Hep/DS synthesis. One study showed that hyaluronic acid levels for type 2 diabetes were higher and that these levels could be used as a biomarker (S. Mine, Y. Okada, C. Kawahara, T. Tabata, Y. Tanaka, Serum hyaluronan concentration as a marker of angiopathy in patients with diabetes mellitus, Endocr. J. 53 (2006) 761-766). This accumulation of HA has already been reported in the lung in adult respiratory distress syndrome, where it is about six times higher than in control patients (R. Hällgren, T. Samuelsson, T. C. Laurent, J. Modig, Accumulation of hyaluronan (hyaluronic acid) in the lung in adult respiratory distress syndrome, Am. Rev. Respir. Dis. 139 (1989) 682-687).

A 2010 study also showed that there is an approximately 66% increase in D-glucuronic acid in the blood of diabetics compared to non-diabetics. Another consequence is a decrease in the activity of xylosyltransferase enzymes (XYLT1, XYLT2) due to a reduction in the xylose attachment positions (caused by a lack of D-xylose or by virus glycosylation at these positions). Indeed, Götting et al, in a study of 100 diabetic patients (Type 1 and Type 2) and 100 blood donations from non-diabetics, demonstrated that serum xylosyltransferase levels in diabetic patients were significantly lower than those in non-diabetic patients. These researchers concluded that serum xylosyltransferase activity could be used as a biomarker of reduced GAG biosynthesis in diabetics (C. Götting, J. Kuhn, K. Kleesiek, Serum xylosyltransferase activity in diabetic patients as a possible marker of reduced proteoglycan biosynthesis, Diabetes Care 31 (2008) 2018-2019).

Additionally, a study on rats showed that such a variation in the activity of the xylosyltransferase 2 enzyme induced lung injuries (R. Koslowski, U. Pfeil, H. Fehrenbach, M. Kasper, E. Skutelsky, K. W. Wenzel, Changes in xylosyltransferase activity and in proteoglycan deposition in bleo-mycin-induced lung injury in rat, Eur. Respir. J. 18 (2001) 347-356).

This further confirms the anti-inflammatory properties of D-xylose and provides an explanation of the process leading to inflammation during diabetes (S. Tsalamandris, A. S. Antonopoulos, E. Oikonomou, G. A. Papamikroulis, G. Vogiatzi, S. Papaioannou, S. Deftereos, D. Tousoulis, The role of inflammation in diabetes: current concepts and future perspectives, Eur. Cardiol. 14 (2019) 50-59).

The antiviral properties of D-xylose confirmed by the tests carried out within the framework of the present invention, corroborate the binding-sites of the SARS-CoV-2 virus using core proteins.

Based on earlier studies, it was explained why the anti-glycemic properties of D-xylose were not bound to insulin, but that the reverse was possible. Since insulin increases the penetration rate of D-xylose into the cells by two to five times, and at equilibrium, in the presence of insulin, xylose is present at 80% instead of 50%-55% in cytosol, and 20% in plasma (Kipnis, D. M, 1957).

The inability of insulin to reduce blood sugar levels is referred to as insulin resistance, and is widely encountered in viral infections of viruses that use proteoglycan (PG) core proteins as cell surface receptors, such as SARS-CoV-2, during diabetes in general.

The notion of insulin resistance thus reflects an underestimation of the storage capacity (weight) of GAGs containing D-xylose with respect to the liver's capacity to store glycogen.

When the liver is full of glycogen and the body does not have enough D-xylose molecules to stimulate HS/CS/DS biosynthesis, any insulin supplementation no longer lowers blood glucose levels, resulting in the situation referred to as insulin resistance. Indeed, in this condition, GAGs do not act as “reservoirs” of glucose metabolites, and since the other reservoir (the liver, where glycogen is stored) is full, insulin supplementation will have no effect on blood glucose levels.

Thus, since xylosyltransferase enzyme activity is reduced during T2DM, the increase in free D-xylose molecules makes it possible to maintain GAG biosynthesis during insulin resistance and thus to reduce blood glucose levels. All the more so as glucose is a competitive inhibitor of the entry of D-xylose into the cell (where GAGs are biosynthesized in the Golgi apparatus).

Glycated hemoglobin or glycosylated hemoglobin (HbA1c) is one of the biomarkers of hyperglycemia in diabetes. It is used for assessment, for long-term control of diabetes and for diabetes diagnosis. The action of D-xylose or its direct metabolite xylitol on glycated hemoglobin (HbA1c) levels has already been the subject of several previous studies, which have shown that taking D-xylose or its direct metabolite xylitol has little or no effect on glycated hemoglobin levels in a healthy person (Bae Y. J. et al, PMID: 22259678; Bordier V. et al, PMID: 34836205).

Viruses Using Syndecans and/or Glypicans as Cell Receptors, (HSV-1, HSV-2, HPV-16, HPV-31, HVB, HVC, HIV-1, HTLV-1, SARS-CoV-2, HCMV, DENV-1, and DENV-2) and Mechanism of Action

The viruses interact with the proteoglycans (PGs) on the surface of the host cells to attach to them (A. Jinno, et al. Methods Mol. Biol. 1229 (2015) 567-585; V. Cagno, et al. Viruses 11 (2019) 596). Several studies have focused on these interactions, in particular on the surface molecules, which are the glycosaminoglycans (GAGs) and heparan sulfate (HS) (A. Jinno, et al. Methods Mol. Biol. 1229 (2015) 567-585; V. Cagno, et al. Viruses 11 (2019) 596). However, even when the viruses interact with HS, the core proteins of most viruses are the viral receptors to which HS fixes covalently (Table 1). This indicates that the interaction observed between HS and certain viruses is due to their binding to the same elements: the core proteins (Table 1). Many of these viruses use other molecules as co-receptors in addition to the core proteins, as is the case with SARS-CoV-2, which uses syndecans and angiotensin-converting enzyme 2 (ACE 2) as co-receptors (M. Bermejo-Jambrina, J. Eder, et al., bioRxiv (2020)). HIV-1 also uses syndecans and CD4 as receptors (A. C. Saphire, et al., J. Virol. 75 (2001) 9187-9200). Hepatitis C virus (HCV) has several other co-receptors in addition to the syndecans (Q. Shi, et al., J. Virol. 87 (2013) 6866-6875).

The different possibilities for post-translational modifications (PTMs) of cell surface proteins may explain the ability of the viruses to use different receptors (J. Hu, et al. Front. Microbiol. 11 (2020), 517461), in particular considering the diversity of glycoproteins present on the viral envelopes and the ability of the viruses to interact with the host target proteins and to use these proteins to enter the cell (S. Maya & A. Ploss, Hepatology 71 (2020) 380-382; K. Azarm, Icahn School of Medicine at Mount Sinai, New York, NY, 2020).

Bermejo-Jambrina et al. (M. Bermejo-Jambrina, J. Eder, et al., bioRxiv (2020)) have recently shown in an in vitro study that SARS-CoV-2 first fixes to heparan sulfate proteoglycans (HSPGs) before interacting with ACE2. However, a recent paper by Clausen et al. (T. M. Clausen et al. Cell 183 (2020) 1043-1057) showed that the SARS-CoV-2 spike protein could bind simultaneously to the cell surface via the HSPGs and the ACE2 protein receptor. The consensus of these cited studies is that the HSPGs are necessary for the attachment of SARS-CoV-2 to the cell surface. Zhang et al. (Q. Zhang, et al, Cell Discov 6 (2020), 80) also came to the same conclusion after an in vitro study.

This is also the case for HIV-1. Saphire et al. (A. C. Saphire, et al., J. Virol. 75 (2001) 9187-9200) have shown in an in vitro study that the CD4 alone are insufficient for HIV-1 infection of macrophages, and that attachment to the HSPGs is also necessary.

TABLE 1
Viruses, core proteins and their associations with type 2 diabetes.

| | Basic proteins of | |
|---|---|---|
| | heparan sulfate | Association with |
| Virus | proteoglycans | diabetes |
| SARS-CoV-2: severe | Syndecan 1 and 4 as | SARS-CoV-2-induced |
| acute respiratory | receptors [(M. | diabetes (F. Rubino et |
| syndrome (SARS) | Bermejo-Jambrina, J. | al., N. Engl. J. Med. |
| coronavirus 2 | Eder, et al., bioRxiv | 383 (2020) 789-790); T. |
| | (2020); A. Hudak et al. | Sathish et al. Prim. |
| | Res. Sq, 2020] | Care Diabetes 15 |
| | | (2020) 194). |
| SARS-CoV | The HSPGs are | |
| | preliminary attachment | |
| | sites for SARS-CoV (J. | |
| | Lang, et al. PLoS One | |
| | 6 (2011)). | |
| HCoV-NL63: human | The HSPGs are | |
| coronavirus NL63 | essential for HCoV- | |
| | NL63 binding (A. | |
| | Milewska, et al. J. Virol. | |
| | 88 (2014) 13221- | |
| | 13230) | |
| HSV-1: herpes | Syndecans 1 and 2 as | Strong association |
| simplex | receptors (S. Bacsa, et | between HSV-1 |
| virus type 1 | al. J. Gen. Virol. | infection and type 2 |
| | 92 (2011) 733-743). | diabetes (Y. Sun et al. |
| | | Diabetes Care 28 (2) |
| | | (2005) 435-436) |
| HPV-16: human | Syndecan 1 as a | Poor prognosis |
| papillomavirus | receptor (Z. Surviladze | (A. Sobti, et al. PLoS |
| type 16 | et al J. Gen. Virol. 96 | One 14 (2019),). |
| | (2015) 2232) | |
| HCV: hepatitis | Syndecan 1 as a | Strong association |
| C virus | receptor (Q. Shi, et al., | between HCV infection |
| | J. Virol. 87 (2013) | and higher prevalence |
| | 6866-6875). | of type 2 diabetes (S. S. |
| | | Hammerstad, et al. |
| | | Front. Endocrinol. 6 |
| | | (2015) 134) |
| HIV-1: human | Syndecans and | HIV is a high-risk factor |
| immunodeficiency | betaglycans as | for the prevalence of |
| virus type 1 | receptors (A. C. | type 2 diabetes (A. D. |
| | Saphire, et al., J. Virol. | Duncan, et al. PLoS |
| | 75 (2001) 9187-9200). | One 13 (2018)). |
| HCMV: human | Strong assumption that | Relative risk ratio of up |
| cytomegalovirus | the syndecans are | to 12 for type 2 |
| | CMV virus receptors, | diabetes in people |
| | based on an in vitro | previously exposed to |
| | study (M. Silvestri, et | CMV (B. W. Roberts, I. |
| | al. J. Immunol. 53 | Cech, South. Med. J. |
| | (2001) 282-289). | 98 (2005) 686-692). |
| DENV: dengue | Syndecan 2 as a | Strong association |
| virus | receptor (K. Okamoto, | between diabetes and |
| | et al. J. Gen. Virol. 93 | the severity of dengue |
| | (2012) 761-770). | (M. Aamir, et al. Med. |
| | | Heal. Sci 9 (2015) |
| | | 99-101.) |
| HTLV-1: human | The HSPGs are | HTLV-1 relative risk |
| T-cell leukemia | essential for HTLV-1 | ratio greater than 5 for |
| virus type 1 | binding (K. S. Jones, et | people with type 2 |
| | al. J. Virol. 79 (2005) | diabetes (M. Taghavi et |
| | 12692-12702). | al. Br. J. Diabetes |
| | | Vasc. Dis. 9 (2009) |
| | | 81-83). |
| HVB: hepatitis | Glypican-5 as a host | HBV is associated with |
| B virus | cell entry factor for | the prevalence of |
| | hepatitis B and D (E. R. | diabetes (Y. S. Hong et |
| | Verrier et al., | al. Sci. Rep. 7 (2017), |
| | Hepatology (Baltimore, | 4606). |
| HVD: hepatitis D virus | MD.) 63 (2016) 35-48). | |

### DEFINITIONS

According to the invention, the term “Formulation” indicates the final presentation of a composition, as it will be given to the patient.

According to the invention, the term “stimulation of glycosaminoglycan biosynthesis” indicates that glycosaminoglycan biosynthesis is higher than it was before administration of the formulation or food supplement according to the invention.

“Glycosaminoglycan” means the family of molecules resulting from the linear polycondensation of osamine and uronic acid units (sometimes replaced by galactose), present essentially in the extracellular matrix of connective tissues. The glycosaminoglycans (GAGs) may or may not be sulfated. There is only one class of non-sulfated GAGs: hyaluronic acid; but there are four classes of sulfated GAGs: chondroitin sulfate, dermatan sulfate, keratan sulfate and heparan sulfate/heparin. Hyaluronic acid is free in tissues, while the sulfated GAGs are attached to carrier proteins (“core proteins”) to form voluminous complexes referred to as proteoglycans. GAGs play an important role in tissue hydration and cell signaling.

“Heparan sulfate” refers to complex polysaccharides belonging to the glycosaminoglycan (GAG) family, abundantly present on cell surfaces and in interstitial matrices. This is a sulfated polyoside of high molecular weight formed by a sequence of α(1-4) linkages of uronic acids and glucosamines, alternating D-glucuronic acid or L-iduronic acid and N-acetylated or N-sulfated glucosamine. This mucopolysaccharide or glycosaminoglycan carries sulfuric residues on the second carbon of the iduronic acids and on the sixth carbon of the glucosamine, as well as on the 2 amine function. This glycan, synthesized in the fibroblasts, is a constituent of the connective tissue (dermis, aorta), forming part of the basement membrane, in which it is bound to collagen and laminin.

“Dermatan sulfate” means a sulfated glycosaminoglycan in which the polyosidic structure does not contain glucuronic acid, but rather L-iduronic acid, which alternates with N-acetylgalactosamine molecules, forming dihexosidic links (L-iduronosido-3-β-N-acetylgalactosamine) attached to C4 of the iduronic acid of the following link. The sulfuric radicals are attached to C4 of the galactosamines. Like most glycosaminoglycans, the dermatan sulfates are part of the glycoprotein building blocks, the proteoglycans, of the connective tissue, particularly the dermis, the cartilage and the cornea. Depending on the tissue, the chains vary in length from 10 to 50 links; they are bound to a polypeptide in the same way as the carbohydrate chains of the glycoproteins. These dermatan sulfates are either localized on the outer surface of the cell membranes, or associated as macromolecular complexes in the extracellular ground substance.

“Chondroitin sulfate” means this sulfated glycosaminoglycan in which the polyosidic structure contains glucuronic acids alternating with N-acetylgalactosamine molecules, forming dihexosidic links (β-glucuronosido-3-β-N-acetylgalactosamine) attached to C4 of the glucuronic acid of the following link. The sulfuric radicals are attached to C4 or C6 of the galactosamines. Like most glycosaminoglycans, the chondroitin sulfates are part of the glycoprotein building blocks, the proteoglycans, of the connective tissue, particularly the dermis, the cartilage and the cornea. Depending on the tissue, the chains vary in length from 10 to 50 links; they are bound to a polypeptide in the same way as the carbohydrate chains of the glycoproteins. These chondroitin sulfates are either located on the outer surface of the cell membranes, or associated as macromolecular complexes in the extracellular ground substance.

“D-xylose esters” means esters, in the sense of the term that is well known to the skilled person, of D-xylose. Preferably, said ester is D-galacturonic acid.

Also advantageously included within the scope of the invention are fatty acid esters comprising 16 to 24 carbon atoms, in particular natural fatty acid esters.

Among these fatty acid esters, esters of palmitic, stearic, oleic, linoleic, linolenic, arachidonic, erucic and lignoceric acids are advantageously selected.

“Oligosaccharides containing D-xylose” mean holosides made up of a small number of doses (2, 3, 4, 5, 6), including D-xylose. For the purposes of the invention, oligosaccharides mean sugar chains containing 2 to 6 sugars.

Preferably, according to the present invention, the xylose-containing oligosaccharide among the oligosaccharides as defined hereinbefore comprises at least one xylose and 1 to 6 sugars.

Such oligosaccharides are advantageously chosen from xylobiose, xylobiose hexaacetate, methyl-β-xylobioside, xylotriose, xylotetraose, xylopentaose and xylohexaose.

Even more preferably, xylobiose is the oligosaccharide used, which consists of two xylose molecules bound by a 1-4 linkage, as well as xylobiose acetates such as xylobiose hexaacetate.

“Human patient” means a human individual suffering from a disease or at risk of suffering from a disease, the prevention or treatment of which is necessary.

“Healthy subject” means a human individual who does not suffer from a disease, and therefore does not require treatment.

“Administering” means the action of the patient or healthy subject taking the formulation or food supplement according to the invention.

“Number of administrations” means the number of times, for example per day, that the formulation or food supplement according to the invention is administered to the patient or healthy subject.

“Acceptable active pharmaceutical ingredient” means an active medicinal substance known to have a particular therapeutic effect.

According to the invention, “antiviral active ingredient” means a compound known for its properties in eliminating viruses, in particular the viruses mentioned in the context of the invention.

According to the invention, “active ingredient for the prevention or treatment of type 2 diabetes” means a compound known for its properties in preventing or treating type 2 diabetes.

According to the invention, the term “active ingredient for the prevention or treatment of insulin resistance” refers to a compound known for its properties in preventing or treating insulin resistance.

According to the invention, “antibiotic” means a compound that acts either by preventing the development of bacteria (bacteriostatic antibiotics), or by killing them (bactericidal antibiotics).

“Dose” means a unit of intake of the formulation or food supplement according to the invention.

“Day” means the unit of 24 hours starting at 00:00 and ending at 23:59.

The “proteoglycans” are glycoproteins consisting of a central protein, also referred to as “carrier protein” or “core protein”, to which one or more chains of sulfated glycosaminoglycans are attached, usually bound by an osidic bond to the alcohol functions of certain serine or occasionally threonine amino acids of the protein. There are currently more than 40 different carrier proteins and five major classes of proteoglycans:
1—small leucin-rich proteoglycans (SLRPs), which stabilize collagen fibers and regulate cellular activity; the main ones are decorin, lumican and fibromodulin;
2—large extracellular proteoglycans, some of which can aggregate with hyaluronic acid to form very large complexes for tissue hydration and shock absorption; the main ones are versican and aggrecan;
3—the proteoglycans associated with the basal membranes, which contribute to tissue cohesion and molecular filtration; the main ones are perlecan and agrin;
4—the proteoglycans of the cell membrane, which play an important role in cell signaling and promote the action of cytokines and growth factors. This class is made up of syndecans, the carrier protein of which is transmembrane, and glypicans, the carrier protein of which is anchored to the cell surface;
5—a small proteoglycan referred to as serglycin found in the intracellular granulations of mast cells and macrophages, which carries chains of heparin, a major anticoagulant.

“Syndecan” means the proteoglycan that is an important component of the membranes of certain cells, such as the sinusoidal membranes of the hepatocytes, muscle cells and macrophages. The syndecans are a family of transmembrane molecules with a glycanic extracellular domain and an intracellular end in the cytoplasm. The syndecans are involved in the internalization of atherogenic lipoproteins.

“Glypican” refers to the proteoglycan in which the carrier protein is anchored to the cell surface.

“HSV-1” is herpes simplex virus type 1.

“HSV-2” is herpes simplex virus type 2.

“HPV-16” is human papillomavirus 16.

“HPV-31” is human papillomavirus 31.

“HVB” is the hepatitis B virus.

“HVC” is the hepatitis C virus.

“HIV-1” is the human immunodeficiency virus.

“HTLV-1” is human T-lymphotropic virus 1.

“SARS-CoV-2” is the virus associated with COVID-19.

“HCMV” is the human cytomegalovirus

“DENV-1” is the first serotype of the dengue virus.

“DENV-2” is the second serotype of the dengue virus.

“Oral administration” or gastrointestinal or per os administration (Latin for “by mouth”) means the enteral route of administration, which consists of swallowing by mouth.

According to the invention, “nasal administration” means the administration of drugs by the nose and the nostrils.

“Parenteral administration” means administration by means of an injection through the skin.

“Subcutaneous” means continuous or discontinuous injection into the subcutaneous tissue (hypodermis).

“Intradermal” means injecting into the skin between the epidermis and the dermis.

“Intravenous” means injecting into a vein.

“Intramuscular” means injecting into a muscle.

According to the invention, the term “preventing” or “prevention” indicates a reduction in the risk of developing severe forms of said diseases.

According to the invention, the term “treating” or “treatment” means alleviating symptoms associated with a specific disorder or condition and/or the elimination of said symptoms.

“Active ingredient for accelerating the transfer of said at least one compound to the cells” means a molecule that facilitates the access of said at least one compound selected from D-xylose, esters thereof, oligosaccharides comprising D-xylose, and/or β-D-xylosides, preferably D-xylose, to target cells through its own action. Preferably, said active ingredient is insulin.

“In a suitable form” means a galenic form enabling the formulation or food supplement according to the invention to be administered correctly, enabling the active substance to reach the target organ as quickly and effectively as possible.

According to the invention, “food supplement” means a product that can provide a nutritional benefit. This supplement can be taken alone or formulated with other compounds to make the composition more attractive to consume by being more similar to a common food product. This supplement can be a factor that contributes to preventing or reducing any superficial visceral pain problems that do not require therapeutic treatment.

The composition according to the invention comprises a “physiologically acceptable medium”, i.e. compatible with oral, nasal or parenteral administration. In other words, the medium used contains compounds that enable the non-degradation of D-xylose or the derivatives thereof, and do not present any risk to the patient or consumer likely to deter them from using this composition, or any risk that could cause them adverse side effects.

In the description and in the following examples, unless otherwise indicated, the percentages are percentages by weight and the ranges of values expressed as “between . . . and . . . ” include the upper and lower limits specified.

The following examples are illustrative and non-limiting of the scope of the invention.

### EXAMPLES

### Example 1: Testing the Antiviral and Cytotoxic Activity of a Compound Against SARS-CoV-2 Using Two Modes of Administration

Three tests were carried out by the VIROLOGY RESEARCH SERVICES (VRS) Laboratory in London to study the cytotoxicity of D-xylose and its antiviral action against SARS-CoV-2 using two different modes of administration
(1) Co-administration of D-xylose and SARS-CoV-2
(2) Pre-incubation of D-xylose 6 h before SARS-CoV-2 infection.

The cytotoxicity of the D-xylose concentrations was determined under the same test conditions, but in the absence of viral infection.

Simultaneous administration of the D-xylose and the virus made it possible to study the competitive inhibitory properties of D-xylose against SARS-CoV-2.

The administration (pre-incubation) of D-xylose 6 h before virus introduction is intended to confirm that the antiviral properties of D-xylose against SARS-CoV-2 are linked to the HS, CS, DS stimulatory action of D-xylose. That is, to confirm that the action of D-xylose on the cell can be transposed to other viruses using the core proteins as receptors.

The 6 h pre-incubation time of the D-xylose administration method (2) was chosen to be in line with the tests that demonstrated the stimulation of HS, CS, DS biosynthesis by D-xylose, in order to dispel any doubts regarding the fact that HS does not promote infection.

### Introduction

An 8-point serial dilution, with a 2-fold dilution factor of D-xylose (highest test concentration: 42 mM) is added to the test. Cells (Vero E6) were used, with two modes of administration: either at the same time or 6 h before infection with SARS-CoV-2 (isolated early).

The compound (D-xylose) was left on the cells for the duration of the experiment (48 h, after which the inhibition of the infection was measured by quantifying the virus-induced cytopathic effect (VIC) by MTT assay. In parallel, the cytotoxicity of the same concentrations of compounds was determined under the same test conditions, but in the absence of viral infection

### Objective

The aim of this study is to test the antiviral and cytotoxic properties of eight concentrations of D(+)-xylose, against an early isolate of SARS-CoV-2, when the compound is administered at the same time or 6 h before infection.

The readout for this test is CPE, so the infection will be stopped at 72 hpi (hours post-infection), and the compound will remain for the duration of the experiment. In parallel, the cytotoxicity of the same compound concentrations will be tested in the absence of viral infection.

### Test Samples

Identity: D(+)-Xylose, Thermo Scientific, AC141001000, Lot: A0429492.
Received: Jul. 14, 2022
Stored: RT

### Controls

Remdesivir

### Test System

Cells: Vero AD
Viruses: England/02/2020, S2P1_July2021, 1.3×107 IU/mL
Readout: CPE (MTT assay) for the antiviral assay & MTT assay for the cytotoxicity assay

### Reagents

Complete media: M199+5% FBS+p/s
Infection media: M199+0.4% BSA+p/2

### Experimental Procedure

### Cellular Veneer

The day before the experiment, the 8,000 cells/well are seeded in two 96-well plates (clear, Sarstedt), one for the antiviral study and one for the cytotoxicity study.

The breakdown of the concentrations is shown in Table 2.

TABLE 2
Plate arrangement

| | | | | Xylose | | | Remdesivir | | | | | | |
|---|---|---|---|---|---|---|---|---|---|---|---|---|---|
| | D-Xylose | | | at the | | | at the | | | | | | |
| D-Xylose | 6 h pre- | | | time of | | | time of | | | | Non- | | Line |
| (mM) | treatment | | | infection | | | infection | | | Infected | infected | Empty | numbers |
| 42 | | | | | | | | | | | | | A |
| 21 | | | | | | | | | | | | | B |
| 10.5 | | | | | | | | | | | | | C |
| 5.25 | | | | | | | | | | | | | D |
| 2.625 | | | | | | | | | | | | | E |
| 1.3125 | | | | | | | | | | | | | F |
| 0.65625 | | | | | | | | | | | | | G |
| 0.328125 | | | | | | | | | | | | | H |
| Column | 1 | 2 | 3 | 4 | 5 | 6 | 7 | 8 | 9 | 10 | 11 | 12 | |
| number | | | | | | | | | | | | | |

Treatment with Compound

A 1 M solution is prepared by dissolving 1 g of xylose in 6.7 ml of sterile water of molecular biology grade [Xylose MW=150.13].

The composition is filtered and sterilized through a 0.22 μM filter.

134.4 μl of the 1 M composition is transferred to 1,465.6 μl of infection medium (=84 mM, twice the final maximum concentration).

The amount of diluent (water) in the mixture is 8.4% (4.2% final).

The “infection medium+diluent” is then prepared by adding 1,680 μl of water to 18,320 μl of infection medium.

For remdesivir: add 3.2 μl of 10 mM stock solution to 796.8 μl of infection medium.

The wells are distributed as follows:

Add 110 μl of infection medium+diluent to wells B to H (rows) in columns 1 to 11.

Add 110 μl of undiluted infection medium to wells B to H (rows) in column 12.

Starting from row A, in order to fill for the lower concentrations, a series of 2-fold dilutions is performed, starting from 110 μl

Next, the cell support in the cytotoxicity plate is removed.

Immediately afterwards, 50 μl of undiluted infection medium is added to all the wells.

Next, 50 μl of infection medium without diluent is added to columns 4 to 6.

Next, 50 μl of diluted compounds are added to the other wells (xylose in 1-3, remdesivir in 7-9, media in 10-12).

Next, the plate is incubated at 37 °C, 5% CO2 for 48 h.

The cell support in the antiviral plate is then removed.

Immediately afterwards, 50 μl of undiluted infection medium is added to all the wells.

Next, 50 μl of infection medium without diluent is added to columns 4 to 12.

Next, 50 μl of diluted compounds are added to columns 1 to 3.

Next, the plate is incubated at 37 °C, 5% CO2 for 6 h.

Next, the dilution plate is stored at 4 °C.

### Infection

Virus required: 16,000×150 wells×MOI 0.002=4,800 IU.

An aliquot of virus is thawed and diluted to 1:10 by adding 10 μl to 90 μl of infection medium.

Next, 4 μl of diluted virus is added to 7.5 ml of infection medium (without diluent).

6 hours after treatment with xylose, the media/composition are removed from the antiviral plate.

50 μl of the dilution plate composition is added to each well.

50 μl of medium is then immediately added to column 11 and to half of column 12.

50 μl of diluted virus is then immediately added to all the other wells.

The plate is then incubated for 48 h, or until the CPE is evident.

### Fixation and Development

After 72 h, when the CPE is evident, an MTT assay is performed for the antiviral and cytotoxicity plates.

### Test Results.

Results for cytotoxicity: absorbance at 570 nm

TABLE 3

| | D-Xylose | | | D-Xylose | | | Remdesivir at | | | | Non- | |
|---|---|---|---|---|---|---|---|---|---|---|---|---|
| D-xylose | 6 h pre- | | | at time of | | | the time of | | | Infected | infected | |
| (mM) | treatment | | | infection | | | infection | | | untreated | untreated | Empty |
| 42 | 0.216 | 0.22 | 0.207 | 0.19 | 0.194 | 0.185 | 0.202 | 0.189 | 0.187 | 0.192 | 0.197 | 0.174 |
| 21 | 0.22 | 0.221 | 0.22 | 0.213 | 0.208 | 0.21 | 0.211 | 0.213 | 0.214 | 0.208 | 0.218 | 0.217 |
| 10.5 | 0.219 | 0.222 | 0.213 | 0.215 | 0.229 | 0.215 | 0.22 | 0.224 | 0.245 | 0.23 | 0.24 | 0.226 |
| 5.25 | 0.231 | 0.238 | 0.222 | 0.227 | 0.238 | 0.224 | 0.224 | 0.216 | 0.231 | 0.214 | 0.191 | 0.191 |
| 2.625 | 0.216 | 0.237 | 0.23 | 0.224 | 0.232 | 0.209 | 0.207 | 0.222 | 0.226 | 0.2 | 0.183 | 0.139 |
| 1.3125 | 0.244 | 0.249 | 0.236 | 0.218 | 0.224 | 0.228 | 0.205 | 0.224 | 0.217 | 0.225 | 0.165 | 0.182 |
| 0.65625 | 0.182 | 0.202 | 0.217 | 0.211 | 0.215 | 0.209 | 0.213 | 0.201 | 0.2 | 0.22 | 0.182 | 0.174 |
| 0.328125 | 0.261 | 0.24 | 0.231 | 0.193 | 0.209 | 0.223 | 0.201 | 0.204 | 0.211 | 0.177 | 0.163 | 0.122 |

### Analysis (Performed by VRS)

Absorbance data

TABLE 4

| | | D-Xylose | | | Remdesivir at | | | | Non- | |
|---|---|---|---|---|---|---|---|---|---|---|
| Remdesivir | D-xylose | 6 h pre- | | | the time of | | | Infected | infected | |
| (μm) | (mM) | treatment | | | infection | | | untreated | untreated | Empty |
| 20 | 42 | 0.216 | 0.22 | 0.207 | 0.202 | 0.189 | 0.187 | 0.192 | 0.197 | 0.174 |
| 10 | 21 | 0.22 | 0.221 | 0.22 | 0.211 | 0.213 | 0.214 | 0.208 | 0.218 | 0.217 |
| 5 | 10.5 | 0.219 | 0.222 | 0.213 | 0.22 | 0.224 | 0.245 | 0.23 | 0.24 | 0.226 |
| 2.5 | 5.25 | 0.231 | 0.238 | 0.222 | 0.224 | 0.216 | 0.231 | 0.214 | 0.191 | 0.191 |
| 1.25 | 2.625 | 0.216 | 0.237 | 0.23 | 0.207 | 0.222 | 0.226 | 0.2 | 0.183 | 0.139 |
| 0.63 | 1.3125 | 0.244 | 0.249 | 0.236 | 0.205 | 0.224 | 0.217 | 0.225 | 0.165 | 0.182 |
| 0.31 | 0.65625 | 0.182 | 0.202 | 0.217 | 0.213 | 0.201 | 0.2 | 0.22 | 0.182 | 0.174 |
| 0.16 | 0.328125 | 0.261 | 0.24 | 0.231 | 0.201 | 0.204 | 0.211 | 0.177 | 0.163 | 0.122 |

Average for the infected untreated column and the infected untreated column: 0.2003125

Pearson's standard deviation for the infected untreated column and the infected untreated column: (Standard deviation: 0.0230903)

### Viability Percentages

TABLE 5

| | | D-Xylose | | | Remdesivir at | | | | Non- | |
|---|---|---|---|---|---|---|---|---|---|---|
| Remdesivir | D-xylose | 6 h pre- | | | the time of | | | Infected | infected | |
| (μm) | (mM) | treatment | | | infection | | | untreated | untreated | Empty |
| 20 | 42 | 107.83 | 109.83 | 103.34 | 100.84 | 94.35 | 93.35 | 95.85 | 98.35 | 86.86 |
| 10 | 21 | 109.83 | 110.33 | 109.83 | 105.34 | 106.33 | 106.83 | 103.84 | 108.83 | 108.33 |
| 5 | 10.5 | 109.33 | 110.83 | 106.33 | 109.83 | 111.83 | 122.31 | 114.82 | 119.81 | 112.82 |
| 2.5 | 5.25 | 115.32 | 118.81 | 110.83 | 111.83 | 107.83 | 115.32 | 106.83 | 95.35 | 95.35 |
| 1.25 | 2.625 | 107.83 | 118.32 | 114.82 | 103.34 | 110.83 | 112.82 | 99.84 | 91.36 | 69.39 |
| 0.63 | 1.3125 | 121.81 | 124.31 | 117.82 | 102.34 | 111.83 | 108.33 | 112.32 | 82.37 | 90.86 |
| 0.31 | 0.65625 | 90.86 | 100.84 | 108.33 | 106.33 | 100.34 | 99.84 | 109.83 | 90.86 | 86.86 |
| 0.16 | 0.328125 | 130.30 | 119.81 | 115.32 | 100.34 | 101.84 | 105.34 | 88.36 | 81.37 | 60.90 |

### Cytotoxicity Percentages

TABLE 6

| | | D-Xylose | | | Remdesivir at | | | | | |
|---|---|---|---|---|---|---|---|---|---|---|
| Remdesivir | D-xylose | 6 h pre- | | | the time of | | | | Non- | |
| (μm) | (mM) | treatment | | | infection | | | Infected | infected | Empty |
| 20 | 42 | −7.83 | −9.83 | −3.34 | −0.84 | 5.65 | 6.65 | 4.15 | 1.65 | 13.14 |
| 10 | 21 | −9.83 | −10.33 | −9.83 | −5.34 | −6.33 | −6.83 | −3.84 | −8.83 | −8.33 |
| 5 | 10.5 | −9.33 | −10.83 | −6.33 | −9.83 | −11.83 | −22.31 | −14.82 | −19.81 | −12.82 |
| 2.5 | 5.25 | −15.32 | −18.81 | −10.83 | −11.83 | −7.83 | −15.32 | −6.83 | 4.65 | 4.65 |
| 1.25 | 2.625 | −7.83 | −18.32 | −14.82 | −3.34 | −10.83 | −12.82 | 0.16 | 8.64 | 30.61 |
| 0.63 | 1.3125 | −21.81 | −24.31 | −17.82 | −2.34 | −11.83 | −8.33 | −12.32 | 17.63 | 9.14 |
| 0.31 | 0.65625 | 9.14 | −0.84 | −8.33 | −6.33 | −0.34 | 0.16 | −9.83 | 9.14 | 13.14 |
| 0.16 | 0.328125 | −30.30 | −19.81 | −15.32 | −0.34 | −1.84 | −5.34 | 11.64 | 18.63 | 39.10 |

### Antiviral Assay Results

### Raw Data:

Absorbances Filter 1: 570 nm

TABLE 7

| | D-Xylose | | | D-Xylose | | | Remdesivir at | | | | | |
|---|---|---|---|---|---|---|---|---|---|---|---|---|
| D-xylose | 6 h pre- | | | at time of | | | the time of | | | | Non- | |
| (mM) | treatment | | | infection | | | infection | | | Infected | infected | Empty |
| 42 | 0.095 | 0.09 | 0.108 | 0.059 | 0.095 | 0.09 | 0.161 | 0.155 | 0.156 | 0.062 | 0.163 | 0.058 |
| 21 | 0.116 | 0.109 | 0.098 | 0.111 | 0.151 | 0.113 | 0.169 | 0.199 | 0.188 | 0.08 | 0.183 | 0.059 |
| 10.5 | 0.102 | 0.102 | 0.096 | 0.109 | 0.131 | 0.107 | 0.198 | 0.215 | 0.174 | 0.086 | 0.155 | 0.058 |
| 5.25 | 0.087 | 0.106 | 0.098 | 0.09 | 0.137 | 0.126 | 0.178 | 0.195 | 0.166 | 0.098 | 0.16 | 0.05 |
| 2.625 | 0.075 | 0.121 | 0.124 | 0.141 | 0.113 | 0.103 | 0.127 | 0.137 | 0.128 | 0.116 | 0.196 | 0.17 |
| 1.3125 | 0.08 | 0.099 | 0.091 | 0.096 | 0.094 | 0.091 | 0.094 | 0.116 | 0.109 | 0.09 | 0.161 | 0.146 |
| 0.65625 | 0.08 | 0.081 | 0.085 | 0.096 | 0.099 | 0.101 | 0.084 | 0.101 | 0.098 | 0.088 | 0.181 | 0.148 |
| 0.328125 | 0.067 | 0.073 | 0.088 | 0.088 | 0.087 | 0.088 | 0.086 | 0.077 | 0.078 | 0.079 | 0.181 | 0.175 |

### Analysis (Performed by VRS)

### Absorbance

TABLE 8

| | | D-Xylose | | | D-Xylose | | | Remdesivir at | | | | Non- | | |
|---|---|---|---|---|---|---|---|---|---|---|---|---|---|---|
| Remdesivir | D-xylose | 6 h pre- | | | at time of | | | the time of | | | Infected | infected | | |
| (μm) | (mM) | treatment | | | infection | | | infection | | | untreated | Untreated | Empty | |
| 20 | 42 | 0.095 | 0.090 | 0.108 | 0.059 | 0.095 | 0.090 | 0.161 | 0.155 | 0.156 | 0.062 | 0.163 | 0.058 | Untreated |
| 10 | 21 | 0.116 | 0.109 | 0.098 | 0.111 | 0.151 | 0.113 | 0.169 | 0.199 | 0.188 | 0.080 | 0.183 | 0.059 | medium |
| 5 | 10.5 | 0.102 | 0.102 | 0.096 | 0.109 | 0.131 | 0.107 | 0.198 | 0.215 | 0.174 | 0.086 | 0.155 | 0.058 | |
| 2.5 | 5.25 | 0.087 | 0.106 | 0.098 | 0.090 | 0.137 | 0.126 | 0.178 | 0.195 | 0.166 | 0.098 | 0.160 | 0.050 | |
| 1.25 | 2.625 | 0.075 | 0.121 | 0.124 | 0.141 | 0.113 | 0.103 | 0.127 | 0.137 | 0.128 | 0.116 | 0.196 | 0.170 | Non- |
| 0.63 | 1.3125 | 0.080 | 0.099 | 0.091 | 0.096 | 0.094 | 0.091 | 0.094 | 0.116 | 0.109 | 0.090 | 0.161 | 0.146 | infected |
| 0.31 | 0.65625 | 0.080 | 0.081 | 0.085 | 0.096 | 0.099 | 0.101 | 0.084 | 0.101 | 0.098 | 0.088 | 0.181 | 0.148 | medium |
| 0.16 | 0.328125 | 0.067 | 0.073 | 0.088 | 0.088 | 0.087 | 0.088 | 0.086 | 0.077 | 0.078 | 0.079 | 0.181 | 0.175 | |

Average for the infected untreated column: 0.087
Standard deviation for the infected untreated column: 0.016
Average for the non-infected untreated column: 0.173
Standard deviation for the non-infected untreated column: 0.015

### Viability Percentages

TABLE 9

| | | | | | | | | | | | | Non- | | | |
|---|---|---|---|---|---|---|---|---|---|---|---|---|---|---|---|
| | | D-Xylose | | | D-Xylose | | | Remdesivir at | | | Infected | infected | | Line | |
| Remdesivir | D-xylose | 6 h pre- | | | at time of | | | the time of | | | un- | Un- | | num- | |
| (μm) | (mM) | treatment | | | infection | | | infection | | | treated | treated | Empty | bers | |
| 20 | 42 | 8.96 | 3.08 | 24.23 | −33.33 | 8.96 | 3.08 | 86.49 | 79.44 | 80.62 | −29.81 | 88.84 | −34.51 | A | Un- |
| 10 | 21 | 33.63 | 25.40 | 12.48 | 27.75 | 74.74 | 30.10 | 95.89 | 131.13 | 118.21 | −8.66 | 112.33 | −33.33 | B | treated |
| 5 | 10.5 | 17.18 | 17.18 | 10.13 | 25.40 | 51.25 | 23.05 | 129.96 | 149.93 | 101.76 | −1.62 | 79.44 | −34.51 | C | medium |
| 2.5 | 5.25 | −0.44 | 21.88 | 12.48 | 3.08 | 58.30 | 45.37 | 106.46 | 126.43 | 92.36 | 12.48 | 85.32 | −43.91 | D | |
| 1.25 | 2.625 | −14.54 | 39.50 | 43.02 | 63 | 30.10 | 18.36 | 46.55 | 58.30 | 47.72 | 33.63 | 127.61 | 97.06 | E | Non- |
| 0.63 | 1.3125 | −8.66 | 13.66 | 4.26 | 10.13 | 7.78 | 4.26 | 7.78 | 33.63 | 25.40 | 3.08 | 86.49 | 68.87 | F | in- |
| 0.31 | 0.65625 | −8.66 | −7.49 | −2.79 | 10.13 | 13.66 | 16.01 | −3.96 | 16.01 | 12.48 | 0.73 | 109.99 | 71.22 | G | fected |
| 0.16 | 0.328125 | −23.94 | −16.89 | 0.73 | 0.73 | −0.44 | 0.73 | −1.62 | −12.19 | −11.01 | −9.84 | 109.99 | 102.94 | H | medium |
| | Column | 1 | 2 | 3 | 4 | 5 | 6 | 7 | 8 | 9 | 10 | 11 | 12 | | |
| | number | | | | | | | | | | | | | | |

### Interpretation of Results

The final cytotoxicity results from [Table 6] received from VRS show that the cytotoxicity concentration of D-xylose in the body is greater than 42 mM

For a reason unexplained by VRS, the top wells (row A of the antiviral plate) appear to inhibit less than row B, which could not be due to increased cell death at the edge of the plate where the drug concentration is higher, in view of the cytotoxicity results.

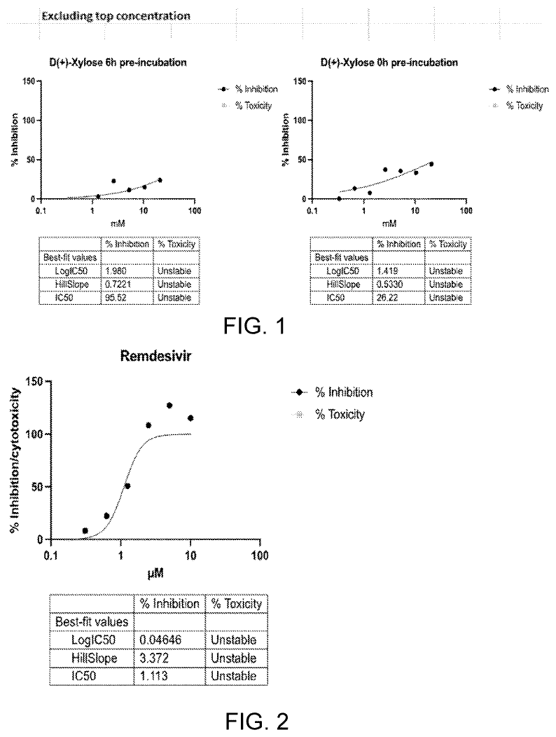

The results were therefore interpreted without the maximum concentration displayed (FIGS. 1 and 2).

A serial dilution with a 2-fold dilution factor was chosen. The same maximum concentration and the same serial dilution were used for both modes of administration of the D-xylose (i.e. simultaneously with the virus or with 6-hour pre-incubation).

Although, as explained above, D-xylose should inhibit SARS-CoV-2 fixing by competitive inhibition, in this first experiment carried out by VRS, it was deliberately chosen to retain the concentrations of D-xylose used to stimulate GAGs in the literature, to confirm that, like heparin, D-xylose, which stimulates GAG biosynthesis (HS/CS/DS/Hep), has antiviral properties, thus contradicting the interpretation according to which HS promotes SARS-CoV-2 infection.

Other arguments that came up again and again during the peer review processes of the previous papers, after several rounds, referred to sulfation.

Any explanation linked to any action of heparin sulfation on SARS-CoV-2 viral attachment is refuted by the fact that even with a pre-incubation of 25 minutes prior to infection, heparin inhibits SARS-CoV-2 viral attachment.

Additionally, in this example, D-xylose, which is a small bioactive molecule, contains no sulfate and stimulates GAG biosynthesis by initiating them.

The antiviral properties of D-xylose against SARS-CoV-2 are confirmed by the present tests.

### Example 2: In Vitro Testing of the Antiviral and Cytotoxic Activity of a Compound Alone or in Combination with a Second Compound Against HIV-1

Three further tests were carried out by VIROLOGY RESEARCH SERVICES (VRS) in London to investigate the cytotoxicity of D-xylose alone at higher concentrations than in the first tests (example 1), the cytotoxicity of D-xylose+insulin and the antiviral activities of D-xylose alone and D-xylose+insulin against HIV-1.

The cytotoxicity of D-xylose alone and D-xylose+insulin concentrations was determined under the same test conditions, but in the absence of viral infection.

Simultaneous administration of D-xylose alone or of D-xylose+insulin and the virus makes it possible to study the competitive inhibitory properties of D-xylose against HIV-1 and to investigate the impact of insulin on the antiviral properties of D-xylose.

The choice of leaving the compounds (D-xylose alone or D-xylose+insulin) for the entire incubation time (well over 6 h) was chosen to dispel any doubts regarding the fact that HS does not promote infection.

### Introduction

An 8-point serial dilution, with a 2-fold dilution factor of a small-molecule compound (D-Xylose; highest test concentration: 208 mM), alone or in combination with insulin, added to the test cells (HeLa Tzmbl) at the same time as HIV-1 infection (NL4.3), according to the dilution scheme hereunder:

TABLE 10

| D-xylose (mM) | | | D-Xylose (mM) + insulin (μM) | | |
|---|---|---|---|---|---|
| 208 | 208 | 208 | 208 + 4.8 | 208 + 4.8 | 208 + 4.8 |
| 104 | 104 | 104 | 104 + 2.4 | 104 + 2.4 | 104 + 2.4 |
| 52 | 52 | 52 | 52 + 1.2 | 52 + 1.2 | 52 + 1.2 |
| 26 | 26 | 26 | 26 + 0.6 | 26 + 0.6 | 26 + 0.6 |
| 13 | 13 | 13 | 13 + 0.3 | 13 + 0.3 | 13 + 0.3 |
| 6.5 | 6.5 | 6.5 | 6.5 + 0.15 | 6.5 + 0.15 | 6.5 + 0.15 |
| 3.25 | 3.25 | 3.25 | 3.25 + 0.075 | 3.25 + 0.075 | 3.25 + 0.075 |
| 1.625 | 1.625 | 1.625 | 1.625 + 0.0375 | 1.625 + 0.0375 | 1.625 + 0.0375 |

The compounds (D-xylose alone or D-xylose+insulin) were left on the cells for the duration of the experiment (48 h, after which inhibition of infection was measured by quantifying the percentage of infected cells using an immunofluorescence-based assay.

In parallel, the cytotoxicity of the same concentrations of compounds was determined under the same test conditions but in the absence of viral infection, using the MTT assay.

### Objective

The aim of this study is to test the antiviral and cytotoxic properties of eight concentrations of D(+)-xylose alone or in combination with eight concentrations of recombinant human insulin against HIV-1 NL4-3.

Test samples
Identity: D(+)-xylose (Thermo Scientific, AC141001000, Batch: A0429492), recombinant human insulin with zinc (Gibco 12585014)
Received: Xylose—Jul. 14; 2022; Insulin—Nov. 1, 2022
Stored: Xylose—room temperature; insulin—80 °C

### Controls

3-azido-3-deoxythymidine (AZT) (Sigma-Aldrich A2169)

### Test System

Cells: HeLa TZMBL
Viruses: Human immunodeficiency virus type 1 (HIV-1) NL4-3, VRS stock 31220 (9.4×105 IU/ml)
Readout: IF (Immunofluorescence) for the antiviral assay & MTT assay for the cytotoxicity test

### Reagents

Complete media: DMEM (Gibco 10566016)+10% FBS (Gibco 10500064)+1×p/s (Gibco 15070063)
Infection media: DMEM (Gibco 10566016)+10% FBS (Gibco 10500064)+1×p/s (Gibco 15070063)

### Experimental Procedure

### Cellular Veneer

The day before the experiment, it is necessary to detach HeLa TZMBL with 5 mM EDTA in PBS and to seed 10,000 cells/well in 2×96 wells, including a black Perkin Elmer plate for the antiviral assay and a transparent Sarstedt plate for the cytotoxicity study

The breakdown of the concentrations is shown in Table 2.

TABLE 11
Plate layout

| | D-xylose | | | Xylose(mM) + | | | AZT | | | | Non- | | Line |
|---|---|---|---|---|---|---|---|---|---|---|---|---|---|
| | (mM) | | | Insulin (μM) | | | Control (μM) | | | Infected | infected | Media | numbers |
| | 208 | 208 | 208 | 208 + | 208 + | 208 + | 1.667 | 1.667 | 1.667 | 0.3 | 0.00 | 0.3 | A |
| | | | | 4.8 | 4.8 | 4.8 | | | | | | | |
| | 104 | 104 | 104 | 104 + | 104 + | 104 + | 0.556 | 0.556 | 0.556 | 0.3 | 0.00 | 0.3 | B |
| | | | | 2.4 | 2.4 | 2.4 | | | | | | | |
| | 52 | 52 | 52 | 52 + | 52 + | 52 + | 0.185 | 0.185 | 0.185 | 0.3 | 0.00 | 0.3 | C |
| | | | | 1.2 | 1.2 | 1.2 | | | | | | | |
| | 26 | 26 | 26 | 26 + | 26 + | 26 + | 0.062 | 0.062 | 0.062 | 0.3 | 0.00 | 0.3 | D |
| | | | | 0.6 | 0.6 | 0.6 | | | | | | | |
| | 13 | 13 | 13 | 13 + | 13 + | 13 + | 0.021 | 0.021 | 0.021 | 0.3 | 0.00 | 0.00 | E |
| | | | | 0.3 | 0.3 | 0.3 | | | | | | | |
| | 6.5 | 6.5 | 6.5 | 6.5 + | 6.5 + | 6.5 + | 0.007 | 0.007 | 0.007 | 0.3 | 0.00 | 0.00 | F |
| | | | | 0.15 | 0.15 | 0.15 | | | | | | | |
| | 3.25 | 3.25 | 3.25 | 3.25 + | 3.25 + | 3.25 + | 0.002 | 0.002 | 0.002 | 0.3 | 0.00 | 0.00 | G |
| | | | | 0.075 | 0.075 | 0.075 | | | | | | | |
| | 1.625 | 1.625 | 1.625 | 1.625 + | 1.625 + | 1.625 + | 1.667 | 1.667 | 1.667 | 0.3 | 0.00 | 0.00 | H |
| | | | | 0.0375 | 0.0375 | 0.0375 | | | | | | | |
| Column | 1 | 2 | 3 | 4 | 5 | 6 | 7 | 8 | 9 | 10 | 11 | 12 | |
| number | | | | | | | | | | | | | |

Wells 1 to 6, 10 and 11 contain infection medium+diluent, while wells 7, 8, 9 and 12 contain infection medium without diluent (diluent for AZT control not included).

### Compound Preparation

Preparation of 3M Xylose [MW=150.13; according to Sigma D(+)-Xylose cat #1.08689, solubility=550 g/L or 3.66 M]:
Dissolve 3 g of xylose in the infection medium to a final volume of 6.66 ml. Heat to 37 °C and vortex.
Sterilize by filtering through a 0.22 μM filter.

Prepare 832 mM xylose (=4× maximum final concentration of 208 mM) in 1000 μl:
277.3 μl 3M Xylose+722.7 μl infection medium (vol=1000 μl).

The stock of recombinant human insulin with zinc (insulin) is 4 mg/ml or 688.7 μM [MW=5807.7] in water.

Prepare 19.2 μM insulin (=4× the maximum final concentration of 4.8 μM) in 500 μl:
13.9 μl insulin 688.7 μM+486.1 μl infection medium (vol=500 μl).

The diluent (water) in this solution is 2.8% (the final is 0.7%).

When equal volumes of 832 mM xylose and 19.2 μM insulin are combined, the diluent (water) concentration is 1.4% (2× final 0.7%). Prepare an “infection medium+total diluent” containing double the final diluent concentration in 15 ml:
210 μl water+14,790 μl infection medium (vol=15 ml).

Prepare “infection medium+insulin diluent” to match the diluent in the 19.2 μM insulin stock for addition to xylose alone (=4× final concentration) in 500 μl:
13.9 μl water+486.1 μl infection medium (vol=500 μl).

The AZT stock is 10 mM in DMSO.

Prepare 10 μM AZT (=2× maximum final concentration of 5 μM):
1 μl AZT 10 mM+999 μl infection medium.

As this is the control and has a different diluent (DMSO), and the final diluent concentration is low (0.05%) at the maximum AZT concentration, the diluent is not taken into account.

Label a 96-well round bottom plate to match the plate layout hereinbefore.

Add 120 μl of 832 mM xylose to wells 1 to 6 of row A.

Add 120 μl of “infection medium+insulin diluent” to wells 1 to 3 of row A.

Add 120 μl of 19.2 μM insulin to wells 4 to 6 of row A.

Add 120 μl of “infection medium+total diluent” to wells 1 to 6 of rows B to H.

Add 240 μl of 10 μM AZT to wells 7 to 9 of row A.

Add 120 μl of infection medium to wells 7 to 9 of rows B to H.

Serially dilute the test samples twice by moving 120 μl from row A (wells 1 to 6) to row H, mixing 10 times.

Serially dilute the control samples 3 times, moving 80 μl from row A (wells 7 to 9) down to row H, mixing 10 times.

Add 240 μl of “infection medium+total diluent” to columns 10 and 11.

Add 240 μl of infection medium to column 12.

### Cytotoxicity

Remove the medium from the cells in the cytotoxicity plate.

Replace with 50 μl of compound dilutions of the round bottom plate immediately followed by 50 μl of infection medium.

Incubate for 48 hours at 37 °C and 5% CO2.

### Infection

Calculate the volume of virus stock required using the formula hereunder: [(Number of cells/well*)×(number of wells+>10% of number of excess wells)×MOI]/virus IU/ml*Double the number of cells spread the day before=(20000×110×0.08/9.4×105×=187 μl

Thaw an aliquot of virus and add 187 μl of virus+5.313 ml of infection medium (total volume 110*50 μl=5500 μl).

Remove the cell support from the antiviral plate.

Replace with 50 μl of dilutions of compounds from the round bottom plate immediately followed by 50 μl of diluted virus or infection medium according to the plate layout hereinbefore (Table 2).

Incubate for 48 hours at 37 °C and 5% CO2.

### Fixation and Development

After 48 hours of incubation, proceed with fixation (antiviral assay) or with the MTT assay (cytotoxicity assay). For the MTT assay, add Triton to a column of untreated cells as a control.

### Immunostaining

Stain the antiviral plate with anti-HIV-1 HXB2 gag p24 (NIBSC Centre For AIDS Reagents ARP432), followed by goat anti-rabbit 488 (Thermo Fisher A11034) at 1:800.

### Test Results.

### Results for Cytotoxicity:

TABLE 12

| D-xylose | Insulin | AZT | | | | Xylose + | | | | | | Media + | | |
|---|---|---|---|---|---|---|---|---|---|---|---|---|---|---|
| (mM) | (μM) | (μM) | D-xylose | | | Insulin | | | AZT | | | diluent | Triton | Media |
| 208.000 | 4.800 | 5.000 | 1.288 | 1.339 | 1.241 | 1.237 | 1.255 | 1.247 | 2.36 | 2.337 | 2.402 | 2.647 | 0.040 | 1.288 |
| 104.000 | 2.400 | 1.667 | 2.075 | 2.027 | 2.064 | 2.022 | 1.991 | 1.934 | 2.469 | 2.46 | 2.505 | 2.485 | 0.039 | 2.075 |
| 52.000 | 1.200 | 0.556 | 2.123 | 2.266 | 2.199 | 2.204 | 2.212 | 2.204 | 2.406 | 2.353 | 2.414 | 2.424 | 0.040 | 2.123 |
| 26.000 | 0.600 | 0.185 | 2.196 | 2.137 | 2.158 | 2.142 | 2.117 | 2.152 | 2.301 | 2.341 | 2.329 | 2.425 | 0.039 | 2.196 |
| 13.000 | 0.300 | 0.062 | 2.175 | 2.293 | 2.032 | 2.281 | 2.316 | 2.235 | 2.337 | 2.307 | 2.356 | 2.347 | 0.040 | 2.175 |
| 6.500 | 0.150 | 0.021 | 2.168 | 2.211 | 2.316 | 2.257 | 2.28 | 2.27 | 2.294 | 2.33 | 2.338 | 2.282 | 0.040 | 2.168 |
| 3.250 | 0.075 | 0.007 | 2.286 | 2.266 | 2.242 | 2.198 | 2.23 | 2.242 | 2.278 | 2.29 | 2.333 | 2.241 | 0.040 | 2.286 |
| 1.625 | 0.038 | 0.002 | 2.399 | 2.351 | 2.424 | 2.409 | 2.38 | 2.39 | 2.438 | 2.455 | 2.474 | 2.431 | 0.040 | 2.399 |

### Analysis (Performed by VRS)

Average for the “Media+diluent” column: =2.410
Standard deviation Standard deviation of the “Media+diluent” column: 0.126

### Viability Percentages

TABLE 13

| D-xylose | Insulin | AZT | | | | Xylose + | | | | | | Media + | | |
|---|---|---|---|---|---|---|---|---|---|---|---|---|---|---|
| (mM) | (μM) | (μM) | D-xylose | | | Insulin | | | AZT | | | diluent | Triton | Media |
| 208.000 | 4.800 | 5.000 | 52.66 | 54.81 | 50.67 | 50.51 | 51.27 | 50.93 | 97.88 | 96.91 | 99.65 | 109.99 | 0.01 | 107.67 |
| 104.000 | 2.400 | 1.667 | 85.86 | 83.83 | 85.39 | 83.62 | 82.31 | 79.91 | 102.48 | 102.10 | 104.00 | 103.15 | −0.03 | 102.35 |
| 52.000 | 1.200 | 0.556 | 87.88 | 93.91 | 91.09 | 91.30 | 91.64 | 91.30 | 99.82 | 97.58 | 100.16 | 100.58 | 0.01 | 101.34 |
| 26.000 | 0.600 | 0.185 | 90.96 | 88.47 | 89.36 | 88.68 | 87.63 | 89.11 | 95.39 | 97.08 | 96.57 | 100.62 | −0.03 | 98.98 |
| 13.000 | 0.300 | 0.062 | 90.08 | 95.05 | 84.04 | 94.55 | 96.02 | 92.61 | 96.91 | 95.64 | 97.71 | 97.33 | 0.01 | 98.64 |
| 6.500 | 0.150 | 0.021 | 89.78 | 91.59 | 96.02 | 93.54 | 94.51 | 94.08 | 95.10 | 96.61 | 96.95 | 94.59 | 0.01 | 97.54 |
| 3.250 | 0.075 | 0.007 | 94.76 | 93.91 | 92.90 | 91.05 | 92.40 | 92.90 | 94.42 | 94.93 | 96.74 | 92.86 | 0.01 | 97.54 |
| 1.625 | 0.038 | 0.002 | 99.53 | 97.50 | 100.58 | 99.95 | 98.72 | 99.15 | 101.17 | 101.89 | 102.69 | 100.88 | 0.01 | 105.85 |

### Cytotoxicity Percentages

TABLE 14

| D-xylose | Insulin | AZT | | | | Xylose + | | | | | | Media + | | |
|---|---|---|---|---|---|---|---|---|---|---|---|---|---|---|
| (mM) | (μM) | (μM) | D-xylose | | | Insulin | | | AZT | | | diluent | Triton | Media |
| 208.000 | 4.800 | 5.000 | 47.34 | 45.19 | 49.33 | 49.49 | 48.73 | 49.07 | 2.12 | 3.09 | 0.35 | −9.99 | 99.99 | −7.67 |
| 104.000 | 2.400 | 1.667 | 14.14 | 16.17 | 14.61 | 16.38 | 17.69 | 20.09 | −2.48 | −2.10 | −4.00 | −3.15 | 100.03 | −2.35 |
| 52.000 | 1.200 | 0.556 | 12.12 | 6.09 | 8.91 | 8.70 | 8.36 | 8.70 | 0.18 | 2.42 | −0.16 | −0.58 | 99.99 | −1.34 |
| 26.000 | 0.600 | 0.185 | 9.04 | 11.53 | 10.64 | 11.32 | 12.37 | 10.89 | 4.61 | 2.92 | 3.43 | −0.62 | 100.03 | 1.02 |
| 13.000 | 0.300 | 0.062 | 9.92 | 4.95 | 15.96 | 5.45 | 3.98 | 7.39 | 3.09 | 4.36 | 2.29 | 2.67 | 99.99 | 1.36 |
| 6.500 | 0.150 | 0.021 | 10.22 | 8.41 | 3.98 | 6.46 | 5.49 | 5.92 | 4.90 | 3.39 | 3.05 | 5.41 | 99.99 | 2.46 |
| 3.250 | 0.075 | 0.007 | 5.24 | 6.09 | 7.10 | 8.95 | 7.60 | 7.10 | 5.58 | 5.07 | 3.26 | 7.14 | 99.99 | 2.46 |
| 1.625 | 0.038 | 0.002 | 0.47 | 2.50 | −0.58 | 0.05 | 1.28 | 0.85 | −1.17 | −1.89 | −2.69 | −0.88 | 99.99 | −5.85 |

### Antiviral Assay Results and Analysis (Performed by VRS)

### % Infection

TABLE 15

| | | | | | | | | | | | | | Non- | |
|---|---|---|---|---|---|---|---|---|---|---|---|---|---|---|
| | | | | | | | | | | | | In- | in- | |
| D-xylose | Insulin | AZT | | | | Xylose + | | | | | | fected + | fected + | |
| (mM) | (μM) | (μM) | D-xylose | | | Insulin | | | AZT | | | diluent | diluent | Media |
| 208.000 | 4.800 | 5.000 | 0.78 | 1.21 | 1.34 | 1.24 | 1.02 | 1.11 | 0.44 | 0.47 | 0.39 | 18.32 | 0.15 | 16.11 |
| 104.000 | 2.400 | 1.667 | 14.95 | 13.53 | 14.38 | 14.21 | 13.16 | 13.04 | 0.95 | 0.89 | 0.97 | 19.39 | 0.29 | 16.55 |
| 52.000 | 1.200 | 0.556 | 20.24 | 20.75 | 18.90 | 19.36 | 19.71 | 21.30 | 1.51 | 1.70 | 1.44 | 19.25 | 0.31 | 16.80 |
| 26.000 | 0.600 | 0.185 | 20.61 | 19.38 | 18.63 | 18.74 | 20.07 | 19.79 | 2.76 | 2.59 | 2.55 | 17.99 | 0.37 | 18.06 |
| 13.000 | 0.300 | 0.062 | 20.38 | 18.66 | 19.01 | 18.22 | 17.71 | 18.29 | 5.12 | 5.07 | 4.49 | 17.99 | 0.43 | 0.24 |
| 6.500 | 0.150 | 0.021 | 18.11 | 18.92 | 16.54 | 19.38 | 19.32 | 19.32 | 6.61 | 5.94 | 7.11 | 19.17 | 0.19 | 0.19 |
| 3.250 | 0.075 | 0.007 | 20.42 | 18.16 | 18.89 | 19.27 | 18.83 | 18.68 | 10.29 | 9.89 | 10.74 | 19.56 | 0.40 | 0.28 |
| 1.625 | 0.038 | 0.002 | 20.77 | 20.01 | 21.42 | 20.67 | 18.00 | 19.83 | 13.79 | 13.28 | 13.72 | 18.81 | 0.28 | 0.20 |

Average for the infected+diluent column: 18.81
Standard deviation for the infected+diluent column: 0.63
Average for the non-infected+diluent column: 0.30
Standard deviation for the non-infected+diluent column: 0.10

### Viability Percentages

TABLE 16

| | | | | | | | | | | | | | Non- | | | |
|---|---|---|---|---|---|---|---|---|---|---|---|---|---|---|---|---|
| D- | Insu- | | | | | | | | | | | In- | in- | | Line | |
| xylose | lin | AZT | | | | Xylose + | | | | | | fected + | fected + | | num- | |
| (mM) | (μM) | (μM) | D-xylose | | | Insulin | | | AZT | | | diluent | diluent | Media | bers | |
| 208.000 | 4.800 | 5.000 | 97.44 | 95.08 | 94.40 | 194.95 | 96.13 | 95.66 | 99.26 | 99.09 | 99.55 | 2.64 | 100.84 | 14.59 | A | Un- |
| 104.000 | 2.400 | 1.667 | 20.84 | 28.55 | 23.94 | 24.83 | 30.53 | 31.18 | 96.51 | 96.80 | 96.37 | −3.15 | 100.04 | 12.22 | B | treated |
| 52.000 | 1.200 | 0.556 | −7.72 | −10.48 | −0.50 | −2.99 | −4.87 | −13.45 | 93.50 | 92.46 | 93.88 | −2.37 | 99.95 | 10.87 | C | medium |
| 26.000 | 0.600 | 0.185 | −9.72 | −3.08 | 0.97 | 0.37 | −6.82 | −5.32 | 86.73 | 87.65 | 87.85 | 4.45 | 99.62 | 4.03 | D | |
| 13.000 | 0.300 | 0.062 | −8.49 | 0.82 | −1.07 | 3.19 | 5.94 | 2.79 | 74.00 | 74.22 | 77.36 | 4.42 | 99.34 | 100.35 | E | Non- |
| 6.500 | 0.150 | 0.021 | 3.77 | −0.58 | 12.28 | −3.07 | −2.75 | −2.75 | 65.92 | 69.53 | 63.24 | −1.94 | 100.58 | 100.62 | F | in- |
| 3.250 | 0.075 | 0.007 | −8.72 | 3.50 | −0.45 | −2.50 | −0.11 | 0.70 | 46.06 | 48.19 | 43.59 | −4.07 | 99.47 | 100.12 | G | fected |
| 1.625 | 0.038 | 0.002 | −10.59 | −6.48 | −14.12 | −10.04 | 4.38 | −5.51 | 27.13 | 29.87 | 27.48 | 0.01 | 100.15 | 100.54 | H | medium |
| | Column | | 1 | 2 | 3 | 4 | 5 | 6 | 7 | 8 | 9 | 10 | 11 | 12 | | |
| | number | | | | | | | | | | | | | | | |

### Interpretation of Results

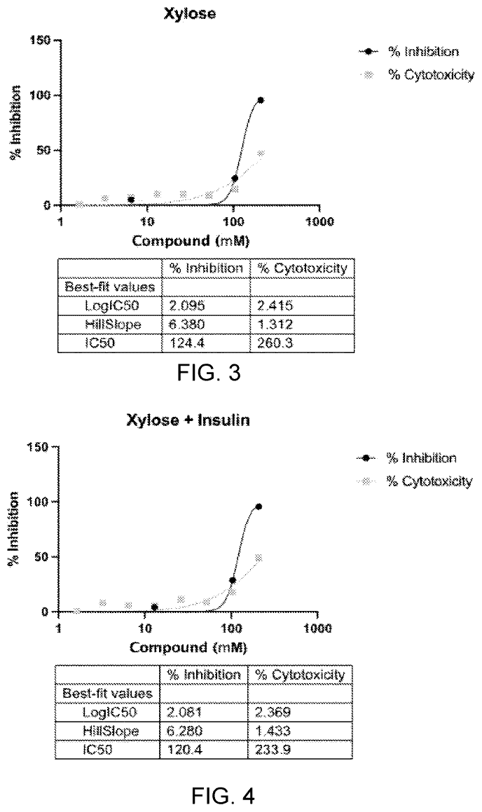

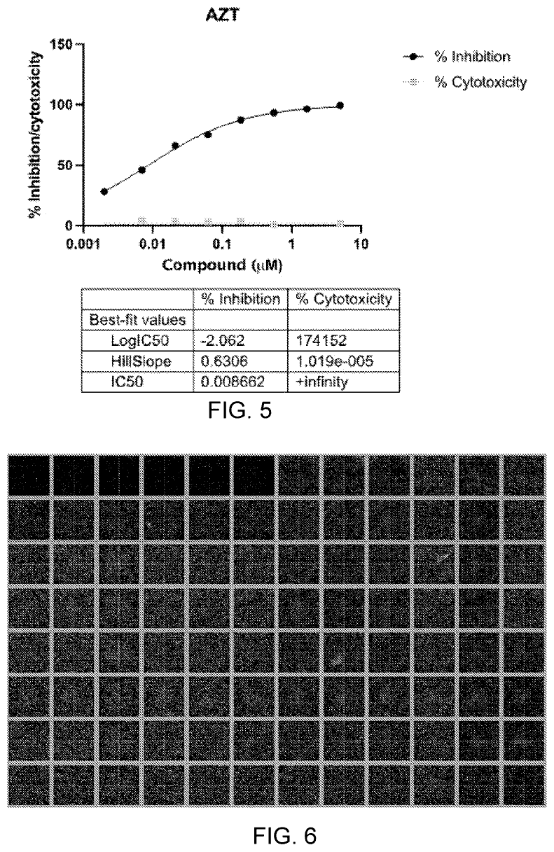

As in example 1, the fact that D-xylose has antiviral activities against HIV-1 NL4-3 confirms the value of focusing on the biosynthesis of glycosaminoglycans, in particular heparan sulfate, chondroitin sulfate and dermatan sulfate, of which D-xylose has been proven to stimulate biosynthesis in order to combat HIV-1 NL4-3.
Calculation of the selectivity index SI=TC50/IC50.
For D-xylose alone against HIV-1, the selectivity index is SI=260.3/124.4=2.092444 (see FIG. 3)
For D-xylose+insulin against HIV-1, the selectivity index is SI=233.9/120.4=1.94269 (see FIG. 4)

The HeLa Tzmbl cell line is an immortal human epithelial cell line derived from a cervical cancer tumor (adenocarcinoma). The question that may arise is whether D-xylose and/or other xylosides stimulate the glycosaminoglycans for all cell types, including epithelial cells, such as the cells used herein. Several D-xylosides, including D-xylose are proven glycosaminoglycan stimulators of several cell types: fibroblasts; keranocytes (epithelial cells); CHO: Chinese hamster ovary cells (epithelial cells); BHK: baby hamster kidney cells; BAE: bovine aortic endothelial cells; SV40-transformed 3T3 Swiss mouse cells; mesenchymal cells; mesenchymal cartilage cells; rat glial cells; mouse neuroblastoma (C1300, NB41A); rat liver cells (HTC, H4), etc.

The antiviral properties of D-xylose against HIV-NL4-3 are confirmed by the present tests.

### Example 3: Summary of the Tests (Examples 1 and 2) Carried Out by the VRS Laboratory in London and Conclusions

### Summary Table

TABLE 17

| | | | | D- | D- | |
|---|---|---|---|---|---|---|
| | | | | xylose | xylose | Selectivity |
| | Method of | | Type of | IC50 | TC50 | index SI = |
| Compound | administration | Virus | cells/tests | (mM) | (mM) | TC50/IC50 |
| D-xylose | Preincubation | SARS- | Vero E6 (CPE& | 95.52 | 260.3 | 2.73 |
| | with cells 6 h | CoV-2 | MTT) | | | |
| | before infection | | | | | |
| | Administration at | SARS- | | 26.22 | 260.3 | 9.93 |
| | the same time as | CoV-2 | | | | |
| | the virus | HIV- | HeLa Tzmbl | 124.4 | 260.3 | 2.09 |
| | | NL4-3 | (Immunofluo- | | | |
| D-xylose + | Administration at | HIV- | rescence & MTT) | 120.4 | 233.9 | 1.94 |
| insulin | the same time as | NL4-3 | | | | |
| | the virus | | | | | |

1. A use of an oral or nasal food supplement composition in healthy subjects comprising D-xylose alone in a physiologically acceptable medium to stimulate the biosynthesis of glycosaminoglycans, preferably at least one selected from heparan sulfate, dermatan sulfate and chondroitin sulfate.

2. The use of an oral or nasal food supplement composition according to claim 1, wherein the composition is in a form suitable for oral or nasal administration by administering between 1 and 10 doses per day of a composition having a mass of 500 mg to 10 g of said at least one compound.

3. The use of an oral or nasal food supplement composition according to claim 2, wherein the number of administrations per day is between 2 and 10 doses per day.

4. The use of an oral or nasal food supplement composition according to claim 1, wherein the D-xylose is present in the food supplement composition in the form of plant extracts comprising D-xylose.

5. The use of an oral or nasal food supplement composition according to claim 4, wherein said plant extracts are selected from extracts of honeysuckle, Japanese honeysuckle, green chiretta, red algae, water bindweed, birch leaf, bark or sap, a species of the genus Artemisia, rice stem.

6. The use of an oral or nasal food supplement composition according to claim 1, wherein the D-xylose is present in the food supplement composition in the form of a lignocellulose derivative of plant materials, preferably hydrolysates of corn stalk hemicellulose.

7. The use of an oral or nasal food supplement composition according to claim 1, wherein said composition is formulated for oral intake in the form of capsules, gelcaps, tablets, effervescent tablets, powders, granules, oral solutions, or suspensions.

8. The use of an oral or nasal food supplement composition according to claim 1, wherein said composition is formulated for oral intake in a food product, beverage, food additive or dairy product, containing said food supplement.

9. The use of an oral or nasal food supplement composition according to claim 1, wherein said composition is formulated for nasal administration as an aerosol solution.
